(19) United States
(12) Patent Application Publication
(10) Pub. No.: US 2025/0345423 A1
(43) Pub. Date: Nov. 13, 2025

(54) BINDING DOMAIN MOLECULES ON CELL SURFACES

(71) Applicant: IMUNEXUS THERAPEUTICS LIMITED, Bundoora, Victoria (AU)

(72) Inventors: George Kopsidas, Preston (AU); Jan Terhag, Preston (AU); Mary-Anne Valerie Schmidt, Craigieburn (AU)

(21) Appl. No.: 18/712,482

(22) PCT Filed: Nov. 25, 2022
(86) PCT No.: PCT/AU2022/051412

§ 371 Date: May 22, 2024

(30) Foreign Application Priority Data
Nov. 26, 2021 (AU) 2021903825

(51) Int. Cl. A61K 40/10; A61K 40/33; A61K 40/42; C07K 14/71; C07K 16/28

(52) U.S. Cl. CPC A61K 40/10; A61K 40/33; A61K 40/4224; C07K 14/71; C07K 16/2818; A61K 2239/13; A61K 2239/21; C07K 2317/567

(57) ABSTRACT

The present disclosure relates to a mammalian cell which is modified to express on the surface of its membrane a binding domain which binds to a target molecule. The disclosure also relates to protein constructs and nucleic acids for producing such modified mammalian cells, and to methods for using the mammalian cells to deliver therapeutic agents to target cells or tissues in vivo.

### FIELD OF THE DISCLOSURE

The present disclosure relates to a mammalian cell which is modified to express on the surface of its membrane a binding domain which binds to a target molecule. The disclosure also relates to protein constructs and nucleic acids for producing such modified mammalian cells, and to methods for using the mammalian cells to deliver therapeutic agents to target cells or tissues in vivo.

### RELATED APPLICATIONS

This application claims priority from AU2021903825 filed 26 Nov. 2021, the entire contents of which are herein incorporated by reference.

### INCORPORATION BY REFERENCE

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

### REFERENCE TO SEQUENCE LISTING

The entire content of the electronic submission of the sequence listing is incorporated by reference in its entirety for all purposes.

### BACKGROUND

The goal of medicine is to maintain, improve or even restore the function of damaged or diseased cells, tissues, and organs. This goal may be achieved by employing cell-based therapeutics to treat patients. However, for the promise of cell-based therapeutics to be fully realized, the cells should migrate or “home” to sites where therapy is needed, and the cells should be capable of providing the therapy desired. Attempts have been made to employ cell-based therapeutics but have met with limited success in a human clinical setting.

Accordingly, there is a substantial need for improved cell-based therapeutics that are able to home to sites in the patient where therapy is desired, and that are able to provide a therapeutic benefit. The present invention addresses these needs and offers other related advantages.

### SUMMARY OF THE DISCLOSURE

In one embodiment the disclosure provides a mammalian cell having a cellular membrane, where the cell is modified to express on the surface of the membrane a cytotoxic T-lymphocyte associated protein (CTLA-4) binding domain which binds to a target molecule.

In one example, binding of the CTLA-4 binding domain to the target molecule homes the cell to the target molecule in vivo.

In one example, binding of the CTLA-4 binding domain to the target molecule homes the target molecule to the cell in vivo.

In one example, the modified mammalian cell is a eukaryotic cell. In another example, the cell is selected from the group consisting of a primate-, monkey- and rodent-derived cell. For example, the cell may belong to any one of the following cell line families: Chinese hamster ovary (CHO), murine myeloma cell (NSO), Human embryonic kidney (HEK293), human myeloma cell line, T cell lymphoma cell line (NOS), kidney fibroblast line (COS), Baby hamster kidney (BHK), HeLa and PER.C6.

In one example, the modified mammalian cell is primary cell. In another example, the cell is selected from the group consisting of a primate-, canine-, feline- and rodent-derived cell. In another example, the primary cell is a neuronal cell, an astrocyte, a fibroblast, a pericyte, a hepatocyte, an osteoblast, an endothelial cell, or an epithelial cell.

Preferably, the cell or cell line is isolated.

In another example, the modified mammalian cell is an immune cell. For example the immune cell may be selected from the group consisting of a T cell (e.g. CD4+), a cytotoxic T cell, a monocyte, a peripheral blood hematopoietic stem cell, a macrophage, an antigen presenting cell, a Natural Killer cell, a mast cell, a neutrophil, an eosinophil, a basophil, a Natural Killer (NK) T cell, a B cell, a dendritic cell, a Helper T cell and a regulatory T cell.

In another example, the modified mammalian cell is a stem cell. In another example, the cell is a pluripotent stem cell. In another example, the stem cell is a mesenchymal lineage precursor or stem cell. In another example, the cell is a mesenchymal precursor cell (MPC) or an mesenchymal stem cell (MSC). In another example, the cell is a differentiated stem cell. For example, the differentiated stem cell may be a differentiated MPC or MSC. In another example, the MPC or MSC is culture expanded.

In one example, the cell is an inducible pluripotent stem cell (IPS).

In one example, the CTLA-4 binding domain comprises or consist of the CTLA-4 sequence set forth in

(SEQ ID NO: 1)
KAMHVAQPAVVLASSRGIASFVCEYASPGKATEVRVTVLRQADSQ
VTEVCAATYMTGNELTFLDDSICTGTSSGNQVNLTIQGLRAMDTG
LYICKVELMYPPPYYLGIGNGTQIYVIDPEPSPDSN.

In one example, the alanine (A) at position 31 of SEQ ID NO:1 is substituted with tyrosine (Y). In one example, the threonine (T) at position 56 of SEQ ID NO:1 is substituted with a methionine (M). In one example, the leucine (L) at position 106 of SEQ ID NO:1 is substituted with a glutamic acid (E).

In one example, the CTLA-4 binding domain comprises or consists of a scaffold having a framework and exposed binding loops (BLs). In one example, the framework corresponds to residues 1 to 25, 34 to 54, 60 to 97 and 106 to 126 of SEQ ID NO:1.

In another example, the CTLA-4 binding domain scaffold comprises or consists of a sequence having at least about 70% sequence identity thereto, or at least 75%, 80%, 85%, 87%, 90%, 92%, 93%, 94%, 95%, 96%, 97% 98% or 99% identity to SEQ ID NO:1 or to residues 1 to 1 to 25, 34 to 54, 60 to 97 and 106 to 126 of SEQ ID NO:1.

In another example, the amino acid residues at positions 26 to 33, and/or positions 55 to 59 and/or positions 98 to 105 of SEQ ID NO:1 are modified or replaced with one or more heterologous sequences.

In another example, the CTLA-4 binding domain comprises or consists of the sequence set forth in:

(SEQ ID NO: 2)
KAMHVAQPAVVLASSRGIASFVCEYXn₁VRVTVLRQADSQVTEVC
AATYXn₂LTFLDDSICTGTSSGNQVNLTIQGLRAMDTGLYICKVX
n₃LGIGNGTQIYVIDPEPSPDSN.

wherein X is any amino acid residue and n is a number between 5 and 15 and n1, n2, n3 indicate binding loops (BLs) 1, 2 and 3 respectively.

In another example, Xn₁ is between 5 and 8 amino acids, Xn₂ is between 5 and 8 amino acids, and Xn₃ is between 10 and 15 amino acids.

In another example a single binding loop, two binding loops or all three binding loops of the native CTLA-4 may be modified by amino acid substitution, addition or deletion, and/or by any change to one or more physical characteristics (e.g. size, shape, charge, hydrophobicity etc.).

In a further example, the exposed binding loop (BL1) sequence ASPGKATE (SEQ ID NO: 3) or ASPGKYTE (SEQ ID NO:4), and/or exposed loop (BL2) sequence MTGNE (SEQ ID NO: 5) and/or the exposed binding loop (BL3) sequence ELMYPPPYY (SEQ ID NO:6) of the CTLA-4 binding domain sequence is modified by amino acid substitution, addition or deletion or replaced with a heterologous sequence.

In one example, the amino acid residues at positions 26 to 33, and/or positions 55 to 59 and/or positions 98 to 105 of SEQ ID NO:1 are modified or replaced. In another example, the amino acid residues at positions 27 to 33, and/or positions 54 to 62 and/or positions 98 to106 of SEQ ID NO:1 are modified or replaced with heterologous sequence.

In another example, the effect of modifying the CTLA-4 binding domain is to abolish its natural affinity to CD80 and CD86.

In one example, the CTLA-4 binding domain scaffold comprises or consists of the sequence:

(SEQ ID NO: 7)
KAMHVAQPAVVLASSRGIASFVCEYXn1VRVTVLRQADSQVTEVC
AATYXn2LTFLDDSICTGTSSGNQVNLTIQGLRAMDTGLYICKVX
n3EGIGNGTQIYVIDPEPSPDSN.

wherein X is any amino acid residue and n is a number between 5 and 15 and the numbers n1, n2 and n3 indicate the binding loop regions. More particularly, 1, 2 and 3 correspond to the BL-1, BL-2 and BL-3 respectively of the CTLA-4 binding domain.

In one example, the BL-1, BL-2 and BL-3 of the CTLA-4 binding domain comprise or consist of respectively ASPGKYTE (SEQ ID NO:4), MTGNE (SEQ ID NO:5) and ELMYPPPYY (SEQ ID NO:6), wherein the domain binds to B7-1.

In one example, the BL-1, BL-2 and BL-3 of the CTLA-4 binding domain comprise or consist of respectively TVSWVDME (SEQ ID NO:8), WNGRW (SEQ ID NO:9) and QLDPSWGYYWQGY (SEQ ID NO:10), wherein the domain binds to sclerostin.

In one example, the CTLA-4 binding domain comprises or consists of the sequence KAMHVAQPAVVLASSRGIASFVCEYASPGKYTEVRVTVLRQADSQVTEVCAATYMTGNELTFL DDSICTGTSSGNQVNLTIQGLRAMDTGLYICKVELMYPPPYYLGIGNGTQIYVIDPEPSPDSN (SEQ ID NO:11), wherein the domain binds to B7-1.

In another example, the CTLA-4 binding domain comprises or consists of the sequence KAMHVAQPAVVLASSRGIASFVCEYTVSWVDMEVRVTVLRQADSQVTEVCAATYWNGRWLT FLDDSICTGTSSGNQVNLTIQGLRAMDTGLYICKVQLDPSWGYYWQGYEGIGNGTQIYVIDPE PSPDSN (SEQ ID NO: 12), wherein the domain binds to sclerostin.

In a further example, BL-1, BL-2 and BL-3 of the CTLA-4 binding domain are replaced with the CDR1, CDR2 and CDR3 sequences respectively of an antibody. The antibody from which the CDR sequences are derived may be derived from any species. In one example, the antibody is derived from a human. In another example, the antibody is derived from a domestic animal, for example, cat, dog, rabbit, guinea pig or horse.

In one example, the CTL-4 binding domain is tethered to the surface of the membrane by a transmembrane domain. Any suitable transmembrane domain may be used. In one example, the transmembrane domain is selected from the group consisting of the transmembrane domain of human platelet-derived growth factor receptor (PDGFR), human asialoglycoprotein receptor, human and murine B7-1, human ICAM-1, human erbb1, human erbb2, human erbb3, human erbb4, human fibroblast growth factor receptors such as FGFR 1, FGFR2, FGFR3, FGFR4, human VEGFR-1, human VEGFR-2, human erythropoietin receptor, human PRL-R, prolactin receptor, human EphA1, Ephrin type-A receptor 1, human insulin, IGF-1 receptors, human receptor-like protein tyrosine phosphatases, human neuropilin, human major histocompatibility complex class II (alpha and beta chains), human integrins (alpha and beta families), human Syndecans, human Myelin protein, human cadherins, human synaptobrevin-2, human glycophorin-A, human Bnip3, human APP, amyloid precursor protein, human T-cell receptor alpha and beta, CD3 gamma, CD3 delta, CD3 zeta, and CD3 epsilon.

In another example, the CTLA-4 binding domain is connected to the transmembrane domain by way of a linker. In one example, the linker comprises a sequence (SGGGG) nS, (SEQ ID NO: 13) wherein n is any number from 2 to 8, or from 3 to 6 or from 3 to 4. In one example, the linker comprises or consists of the sequence SGGGGSGGGGSGGGGS (SEQ ID NO:14) or SGGGGSGGGGSGGGGSGGGGS (SEQ ID NO:15).

In a second embodiment, the disclosure provides a method for homing mammalian cells to a target molecule in a subject, comprising administering to the subject the modified mammalian cell described herein according to the first embodiment.

In one example, the cell has also been modified to carry a therapeutic agent. The therapeutic agent may be, for example, an anticancer agent or an immunomodulatory agent. In another example, the therapeutic agent is a naturally occurring or modified oncolytic virus.

It will be appreciated, however, that the modified mammalian cells of the present disclosure may carry natural therapeutic agents without the need for further modification. For example, MPCs or MSCs naturally contain beneficial proteins, such as cytokines, enzymes and other proteins, which can be delivered to target cells or tissue in vivo via paracrine signalling. These cells may therefore may be considered therapeutic agent themselves, or as adjuvants for co-administered therapeutic agents.

In one example, the target molecule is selected from the group consisting of a target expressed by a tumour and a target associated with the tumour stroma.

In a third embodiment, the disclosure provides a method for homing target molecules to a mammalian cell as described herein, comprising administering to a subject the modified mammalian cell as described herein.

For example, the modified mammalian cell of the present invention may be tethered to or embedded within a tissue site where, for example, regeneration or repair is needed. The tethered cell then binds the target molecule of interest to home that target molecule to the damaged site. Examples of suitable target molecules include growth effector molecules, including growth factors and extracellular matrix molecules, which stimulate and support cell and tissue growth or promote wound healing.

In a fourth embodiment, the disclosure provides a chimeric binding domain comprising:
(a) a leader sequence for translocating the chimeric domain across an intracellular membrane;
(b) a CTLA-4 binding domain specific for a target molecule; and
(c) a transmembrane domain which anchors the chimeric domain to the surface membrane of a mammalian cell.

In one example the chimeric binding domain further comprises a linker sequence located between the CTLA-4 binding domain and the transmembrane domain. In one example, the linker is a peptide linker. Any suitable peptide linker known in the art can be utilised in the present disclosure. In one example, the linker is a Gly-Ser peptide linker.

In one example, the linker comprises a sequence (SGGGG) nS, (SEQ ID NO:13) wherein n is any number from 2 to 8, or from 3 to 6 or from 3 to 4. In one example, the linker comprises or consists of the sequence SGGGGSGGGGSGGGGS (SEQ ID NO:14) or SGGGGSGGGGSGGGGSGGGGS (SEQ ID NO:15).

In one example the leader sequence is selected from the group consisting of Mouse Ig Kappa (METDTLLLWVLLLWVPGSTGD; SEQ ID NO:16), Human OSM (MGVLLTQRTLLSLVLALLFPSMASM; SEQ ID NO:17), VSV-G (MKCLLYLAFLFIGVNC; SEQ ID NO: 18), Human IgG2 H (MGWSCIILFLVATATGVHS; SEQ ID NO:19), BM40 (MRAWIFFLLCLAGRALA; SEQ ID NO:20), Secrecon (MWWRLWWLLLLLLLLWPMVWA; SEQ ID NO: 21), Human IgKVIII (MDMRVPAQLLGLLLLWLRGARC; SEQ ID NO:22), CD33 (MPLLLLLPLLWAGALA; SEQ ID NO:23), tPA (MDAMKRGLCCVLLLCGAVFVSPS; SEQ ID NO: 24), Human Chymotrypsinogen (MAFLWLLSCWALLGTTFG; SEQ ID NO:25), Human trypsinogen-2 (MNLLLILTFVAAAVA; SEQ ID NO:26), Human IL-2 (MYRMQLLSCIALSLALVTNS; SEQ ID NO:27), Gaussia luc (MGVKVLFALICIAVAEA; SEQ ID NO: 28), Albumin (HSA) (MKWVTFISLLFSSAYS; SEQ ID NO:29), Influenza Haemagglutinin (MKTIIALSYIFCLVLG; SEQ ID NO:30), Human insulin (MALWMRLLPLLALLALWGPDPAAA; SEQ ID NO:31), Silkworm Fibroin LC and (MKPIFLVLLVVTSAYA; SEQ ID NO:32).

In one example the transmembrane domain comprises between 17 and 29 residues, or between 19 and 26 residues, or between 21 and 24 residues. In another example, the transmembrane domain is selected from the group consisting of the transmembrane domain of human platelet-derived growth factor receptor (PDGFR), human asialoglycoprotein receptor, human and murine B7-1, human ICAM-1, human erbb1, human erbb2, human erbb3, human erbb4, human fibroblast growth factor receptors such as FGFR 1, FGFR2, FGFR3, FGFR4, human VEGFR-1, human VEGFR-2, human erythropoietin receptor, human PRL-R, prolactin receptor, human EphA1, Ephrin type-A receptor 1, human insulin, IGF-1 receptors, human receptor-like protein tyrosine phosphatases, human neuropilin, human major histocompatibility complex class II (alpha and beta chains), human integrins (alpha and beta families), human Syndecans, human Myelin protein, human cadherins, human synaptobrevin-2, human glycophorin-A, human Bnip3, human APP, amyloid precursor protein, human T-cell receptor alpha and beta, CD3 gamma, CD3 delta, CD3 zeta, and CD3 epsilon.

In a fifth embodiment, the disclosure provides a nucleic acid molecule encoding the chimeric binding domain described herein. In one example, the nucleic acid is DNA, RNA or both.

The present disclosure also provides a nucleic acid encoding a polypeptide of the present disclosure, in particular a polypeptide of any one of SEQ ID NOs: 1, 11 or 12.

In one example, the molecule comprises or consists of the nucleic acid sequence of a binding domain set forth in:

(SEQ ID NO: 33)
AAAGCAATGCATGTTGCACAGCCTGCAGTTGTTCTGGCAAGCAGC
CGTGGTATTGCCAGCTTTGTTTGTGAGTATN1GTGCGCGTGACCG
TTCTGCGTCAGGCAGATAGCCAGGTTACCGAAGTTTGTGCAGCAA
CCTATN2CTGACCTTTCTGGATGATAGCATTTGTACCGGCACCAG
CAGCGGTAATCAGGTTAATCTGACCATTCAGGGTCTGCGTGCAAT
GGATACCGGTCTGTATATTTGCAAAGTTN3CTGGGCATTGGCAAT
GGCACCCAGATTTATGTTATTGATCCTGAACCGTCACCGGATAGC
AATGC

wherein N1 is length of nucleotides encoding a first binding loop, N2 is a length of nucleotides encoding a second binding loop and N3 is a length of nucleotides encoding a third binding loop. In one example, N1, N2 and N2 are between 15 and 45 nucleotides. In one example, N1 is between 15 and 24 nucleotides. N2 is 15 nucleotides and N3 is between 30 and 45 nucleotides. N is any nucleotide (A, C, T, G).

In one example, the nucleic acid is provided in an expression construct in which the nucleic acid is operably linked to a promoter. Such an expression construct can be in a vector e.g. a plasmid.

In a sixth embodiment, the disclosure provides a host cell transformed with a nucleic acid described herein. Suitable host cells include bacteria, mammalian cells, yeast, moss (bryophytes), and baculovirus systems.

In a seventh embodiment, the disclosure provides a pharmaceutical composition comprising the modified mammalian cell described herein, together with a pharmaceutically acceptable carrier and/or excipient. The composition may be provided as a medicament. In one example, the composition is for use in the treatment of a disorder.

In another example, the binding domain may be labelled with an agent to facilitate detection.

It will be appreciated that the mammalian cells and pharmaceutical compositions described herein are suitable for use as a medicament and in the treatment of a wide range of conditions or diseases including, for example, the treatment of oncology disease, autoimmune, neurological diseases, orthopaedic conditions, cardiac disease and traumatic injuries.

### DESCRIPTION OF DRAWINGS

FIG. 1 shows the results of in-cell ELISA assays performed on HEK293 cells transfected with either plasmid DNA expressing a CTLA-4 BD (either BD_B7 or BD_SOST) or no plasmid DNA (mock transfection). Panel A shows the detection of BD_B7 on the cell surface using an anti-CTLA-4 primary antibody (1:1000) and an anti-Mouse-IgG (HRP) secondary antibody (1:5000). Panel B shows the detection of BD_SOST on the cell surface using the same antibodies as in panel A; Panel C shows the detection of human recombinant Sclerostin bound to BD_SOST-expressing HEK cells using an anti-SOST primary antibody (1:1000) and an anti-Mouse-lgG (HRP) secondary antibody (1:5000). All values are arithmetic means±SEM. Asterisks indicate p-values from two-tailed, unpaired t-tests: *p≤0.05; ***p≤0.001.

FIG. 2 shows the results of in-cell ELISA assays performed on CHO cells transfected with either plasmid DNA expressing a CTLA-4 BDM (either BD_B7 or BD_SOST) or no plasmid DNA (mock transfection). Panel A shows the detection of BD_B7 on the cell surface using an anti-CTLA-4 primary antibody (1:1000) and an anti-Mouse-lgG (HRP) secondary antibody (1:5000). Panel B shows the detection of BD_SOST on the cell surface using the same antibodies as in panel A; Panel C shows the detection of human recombinant Sclerostin bound to BD_SOST-expressing HEK cells using an anti-SOST primary antibody (1:1000) and an anti-Mouse-IgG (HRP) secondary antibody (1:5000). All values are arithmetic means±SEM. Asterisks indicate p-values from two-tailed, unpaired t-tests: *p≤0.05; ***p≤0.001.

FIG. 3 shows the results of in-cell ELISA assays performed on adipose tissue-derived transfected with either plasmid DNA expressing a CTLA-4 BDM (either BDM_B7 or BDM_SOST) or no plasmid DNA (mock transfection). Panel A shows the detection of BDM_B7 on the cell surface using an anti-CTLA-4 primary antibody (1:1000) and an anti-Mouse-IgG (HRP) secondary antibody (1:5000). Panel B shows the detection of BDM_SOST on the cell surface using the same antibodies as in panel A; binding of rhSOST could not be assessed since aMSCs endogenously express SOST-binding proteins. All values are arithmetic means±SEM. Asterisks indicate p-values from two-tailed, unpaired t-tests: **p≤0.01; ***p≤0.001.

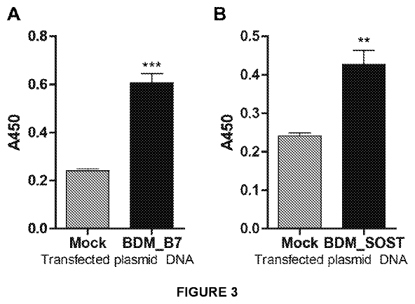

FIG. 4 shows the results of in-cell ELISA assays performed on bone marrow-derived mesenchymal stem cells (bMSCs) transfected with either plasmid DNA expressing a CTLA-4 BD (either BD_B7 or BD_SOST) or no plasmid DNA (mock transfection). Panel A shows the detection of BD_B7 on the cell surface using an anti-CTLA-4 primary antibody (1:1000) and an anti-Mouse-IgG (HRP) secondary antibody (1:5000). Panel B shows the detection of BD_SOST on the cell surface using the same antibodies as in panel A. Panel C shows the detection of human B7-1 (CD80) protein bound to BD_B7-expressing bMSCs using an anti-CD80 primary antibody (1:1000) and an anti-Mouse IgG (HRP) secondary antibody (1:5000); binding of rhSOST could not be assessed since bMSCs endogenously express SOST-binding proteins. Panel D shows the detection of Raji cells bound to BD_B7-expressing bMSCs using an anti-CD80 primary antibody (1:1000) and an anti-Mouse IgG (HRP) secondary antibody (1:5000). Panel E shows immunofluorescent staining of a transfected bMSC expressing BD_B7 stained with an anti-CTLA-4 antibody (green) and nuclear stain DAPI (blue). All values are arithmetic means±SEM. Asterisks indicate p-values from two-tailed, unpaired t-tests: **p≤0.01; ***p≤0.001.

### KEY TO SEQUENCE LISTING

SEQ ID NO 1: amino acid sequence of the CTLA-4 binding domain
SEQ ID NO 2: amino acid sequence of the CTLA-4 binding domain scaffold 1
SEQ ID NO 3: amino acid sequence encoding exposed binding loop (BL1) sequence 1
SEQ ID NO 4: amino acid sequence encoding exposed binding loop (BL1) sequence 2
SEQ ID NO 5: amino acid sequence encoding exposed loop (BL-2) sequence
SEQ ID NO 6: amino acid sequence encoding exposed binding loop (BL-3) sequence
SEQ ID NO 7: amino acid sequence of the CTLA-4 binding domain scaffold 2
SEQ ID NO 8: amino acid sequence encoding sclerostin BL-1 sequence
SEQ ID NO 9: amino acid sequence encoding sclerostin BL-2 sequence
SEQ ID NO 10: amino acid sequence encoding sclerostin BL-3 sequence
SEQ ID NO 11: amino acid sequence encoding CTLA-4 binding domain for B7-1
SEQ ID NO 12: amino acid sequence encoding CTLA-4 binding domain for sclerostin
SEQ ID NO 13: amino acid sequence encoding linker
SEQ ID NO 14: amino acid sequence encoding linker
SEQ ID NO 15: amino acid sequence encoding linker
SEQ ID NO 16: amino acid sequence encoding the leader sequence of Mouse Ig Kappa
SEQ ID NO 17: amino acid sequence encoding the leader sequence of Human OSM
SEQ ID NO 18: amino acid sequence encoding the leader sequence of VSV-G
SEQ ID NO 19: amino acid sequence encoding the leader sequence of Human lgG2 H
SEQ ID NO 20: amino acid sequence encoding the leader sequence of BM40
SEQ ID NO 21: amino acid sequence encoding the leader sequence of Secrecon
SEQ ID NO 22: amino acid sequence encoding the leader sequence of Human IgKVIII
SEQ ID NO 23: amino acid sequence encoding the leader sequence of human CD33
SEQ ID NO 24: amino acid sequence encoding the leader sequence of tPA
SEQ ID NO 25: amino acid sequence encoding the leader sequence of Human
Chymotrypsinogen
SEQ ID NO 26: amino acid sequence encoding the leader sequence of Human trypsinogen-2
SEQ ID NO 27: amino acid sequence encoding the leader sequence of Human IL-2
SEQ ID NO 28: amino acid sequence encoding the leader sequence of Gaussia luc
SEQ ID NO 29: amino acid sequence encoding the leader sequence of Albumin (HSA)
SEQ ID NO 30: amino acid sequence encoding the leader sequence of Influenza Haemagglutinin
SEQ ID NO 31: amino acid sequence encoding the leader sequence of Human insulin
SEQ ID NO 32: amino acid sequence encoding the leader sequence of Silkworm Fibroin LC
SEQ ID NO 33: nucleic acid sequence encoding a CTLA-4 binding domain

### DESCRIPTION OF EMBODIMENTS

### Selected Definitions

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

A “CTLA-4 binding domain” is a domain derived from the N-terminal extracellular domain of CTLA-4. The CTLA-4 binding domain may comprise a native CTLA-4 sequence or a modified version thereof, for example as described in SEQ ID NO:1 with altered C-terminal sequence. The modifications can occur in the framework or scaffold region, or in one or more of the binding loop (BL) sequences. In one example, all BL sequences are replaced by heterologous or random BL sequences so that the CTLA-4 binding domain no longer binds to its native ligands. In one example the binding specificity of the modified CTLA-4 domain is altered so that the domain binds to a different target molecule of interest.

A “binding loop” (BL) is a polypeptide loop structure or region that functions in a similar manner to the complementarity determining regions (CDRs) in antibody variable domains that bind to specific antigens. Three antigen binding loop sequences (referred to herein as BL-1, BL-2 and BL-3 respectively) are present in the CTLA-4 binding domain and they sit within a scaffold sequence which provides the required three-dimensional conformation of the loop sequences. Native BL sequences can be replaced with one or more corresponding CDRs which can be grafted onto the scaffold. Diversity can be introduced into the BL sites of the CTLA-4 binding domain by randomising the amino acid sequence of the specific loops of the scaffold e.g. by introducing NNK codons followed by selection for desired binding characteristics using, for example, display technologies. This mechanism is similar to natural selection of high affinity, antigen-specific antibodies.

The term ‘binding specificity’ in the context of a CTLA-4 binding domain, refers to the ability of the domain to bind its respective target antigen or epitope which is dependent upon the presence of a particular structure (e.g., an antigenic determinant or epitope) on the target antigen or epitope. For example, the CTLA-4 binding domain recognizes and binds to a specific protein structure rather than to proteins generally. By way of example, if the domain binds to epitope “A”, the presence of a molecule containing epitope “A” (or free, unlabelled “A”), in a reaction containing labelled “A” and the domain, will reduce the amount of labelled “A” bound to the domain.

The term should also be understood to include that the CTLA-4 binding domain “specifically binds” to a target antigen. The term ‘specifically binds’ or ‘binds specifically’ shall be taken to mean that the CTLA-4 binding domain of the present disclosure reacts or associates more frequently, more rapidly, with greater duration and/or with greater affinity with a particular target antigen than it does with alternative target antigens. Reference to ‘binding’ provides explicit support for the term ‘specific binding’ and vice versa. Typically, the term is used to describe the affinity of the domain for a given target antigen. In some circumstances, it may be desirable to have low affinity binding where toxicity may be an issue. In other circumstances it may be desirable to have high affinity binding to minimise cross-reactivity to other target antigens. In one example, the binding is specific binding as described herein.

The term “binding affinity” or “affinity” of a moiety of the molecule (i.e. the protein or the BDM) to a selected target can be measured. The term ‘affinity’ refers to the equilibrium constant for the reversible binding of two agents and is expressed as a dissociation constant (Kd) or equilibrium dissociation constant (KD).

The term “target molecule” as used herein means a substance to which the CTLA-4 binding domain binds. The target molecule may be, for example, an antigen. An antigen will typically comprise one or more antigenic epitopes which are recognised by the CTLA-4 binding domain. The protein antigen may be a soluble protein or membrane bound protein. Examples of soluble proteins include, but are not limited to transcription factors, antibodies, growth factors, blood proteins (e.g. albumin), or drugs (e.g. steroid, pharmaceutical drugs etc.). 85asZTypes of membrane bound proteins include growth factor receptors, tumour markers, cell surface markers, or markers which mediate transport into a cell (e.g. transferrin), or Fc receptor. It typically refers to a substance which is capable of raising an immune response in vivo. It may be a polypeptide, protein, nucleic acid (e.g. DNA, RNA or a combination of DNA and RNA) or other molecule.

As used herein, the term “epitope” (syn. “antigenic determinant”) shall be understood to mean a region to which a CTLA-4 binding domain of the present disclosure binds. Conventionally, the term refers to a structure bound by an immunoglobulin VH/VL pair. An epitope defines the minimum binding site for a CTLA-4 binding domain. This term is not necessarily limited to the specific residues or structure to which a CTLA-4 binding domain makes contact. For example, this term includes a region spanning amino acids contacted by the BL sequences of the CTLA-4 binding domain, and 5-10 (or more) or 2-5 or 1-3 amino acids outside of this region. In some examples, the epitope comprises a series of discontinuous amino acids that are positioned close to one another when a polypeptide is folded and, for example, associated with another polypeptide, i.e., a ‘conformational epitope’. The term includes those composed of a linear peptide sequence (i.e., “continuous”) or those composed of non-contiguous amino acid sequences (i.e., “conformational” or “discontinuous”).

An iPS cell as referred to herein is understood to refer to a cell derived from skin or blood which has been reprogrammed back into an embryonic-like pluripotent state that enables the development of an unlimited source of any type of human cell needed for therapeutic purposes.

### CTLA-4 Binding Domains

Cytotoxic T-lymphocyte associated antigen 4 (CTLA-4) is involved in T-cell regulation during the immune response. CTLA-4 is a 44 kDa homodimer expressed primarily and transiently on the surface of activated T-cells, where it interacts with CD80 and CD86 surface antigens on antigen presenting cells to effect regulation of the immune response (Waterhouse et al. (1996) Immunol Rev 153:183-207, van der Merwe et al. (1997) J Exp Med 185 (3): 393-403).

Each CTLA-4 monomeric subunit consists of an N-terminal extracellular domain, transmembrane domain and C-terminal intracellular domain. The extracellular domain comprises an N-terminal V-like domain (VLD; of approximately 14 kDa predicted molecular weight by homology to the immunoglobulin superfamily) and a stalk of about 10 residues connecting the VLD to the transmembrane domain. The VLD comprises surface loops corresponding to BL-1, BL-2 and BL-3 respectively (Metzler W J et al (1997) Nat Struct Biol 4 (7): 527-31) which binds to CD80 and/or CD86. The sequence of human CTLA-4 has been previously determined (U.S. Pat. Nos. 5,434,131; 5,844,095; 5,851,795).

Structural and mutational studies on CTLA-4 suggest that binding to CD80 and CD86 occurs via the VLD surface formed from ‘GFCC’ V-like beta-strands and also from the highly conserved MYPPPYY sequence in the BL-3. Dimerisation between CTLA-4 monomers occurs through a disulphide bond between cysteine residues (Cys120) in the two stalks, which results in tethering of the two extracellular domains, but without any apparent direct association between V-like domains (Metzler W J et al (1997) Nat Struct Biol 4 (7): 527-31). Dimerisation appears to contribute exclusively to increased avidity for the ligands.

The human sequence for CTLA-4 is available as UniProt reference P16410. The extracellular domain of CTLA-4 corresponds to positions 36-161 of the sequence (wherein the CTLA-4 has a total length of 126 amino acids). Amino acid residues 1-35 correspond to the signal peptide.

As shown herein, replacement of one or more binding loop structures in the CTLA-4 binding domain with heterologous binding loop sequences directed against sclerostin or CD3 resulted in the production of soluble, monomeric, unglycosylated binding molecules using a mammalian expression system. The VLDs thus provide a basic framework for constructing soluble, single domain molecules wherein the binding specificity of the molecule may be engineered by modifications of the binding loop structures.

The framework residues of the CTLA-4 binding domain may be modified in accordance with structural features present in camelid antibodies. The camel heavy chain immunoglobulins differ from conventional antibody structures by consisting of a single VH domain.

Several non-conventional substitutions (predominantly hydrophobic to polar in nature) at exposed framework residues reduce the hydrophobic surface, while maintaining the internal beta-sheet framework structure (Desmyter et al. (1996) Nat Struct Biol 3:803-811).

Within the three binding loops several structural features compensate for the loss of antigen binding-surface usually provided by the VLD. While the BL2 loop does not differ extensively from other VH domains, the BL1 and BL3 adopt non-canonical conformations which are extremely heterologous in length. For example, the H1 loop may contain anywhere between 2-8 residues compared to the usual five in Ig molecules. However, it is the BL3 which exhibits greatest variation: in 17 camel antibody sequences reported, the length of this region varies between 7 and 21 residues (Muyldermans et al. (1994) Protein Eng 7:1129-1135). Thirdly, many camelid VH domains possess a disulphide linkage interconnecting BL1 and BL3 in the case of camels and interconnecting CDRs-1 and -2 in the case of llamas (Vu et al. 1997). The function of this structural feature appears to be maintenance of loop stability and providing a more contoured, as distinct from planar, loop conformation which both allows binding to pockets within the antigen and gives an increased surface area. However, not all camelid antibodies possess this disulphide bond suggesting that it is not an absolute structural requirement.

These foregoing features have enabled camelid V-domains to present as soluble molecules in vivo and with sufficiently high affinity to form an effective immune response against a wide variety of target molecules.

Methods for generating and selecting single VLD molecules with novel binding affinities for target molecules have been described in U.S. Pat. No. 7,166,697, the entire contents of which are incorporated by reference. The method involves the application of well-known molecular evolution techniques to VLDs derived from members of the immunoglobulin superfamily. The method may involve the production of phage or ribosomal display libraries for screening large numbers of mutated VLDs.

Filamentous fd-bacteriophage genomes are engineered such that the phage display, on their surface, proteins such as the Ig-like proteins (Fabs) which are encoded by the DNA that is contained within the phage (Smith, 1985; Huse et al., 1989; McCafferty et al., 1990; Hoogenboom et al., 1991). Protein molecules can be displayed on the surface of Fd bacteriophage, covalently coupled to phage coat proteins encoded by gene III, or less commonly gene VIII. Insertion of antibody genes into the gene III coat protein give expression of 3-5 recombinant protein molecules per phage, situated at the ends. In contrast, insertion of antibody genes into gene VIII has the potential to display about 2000 copies of the recombinant protein per phage particle, however this is a multivalent system which could mask the affinity of a single displayed protein. Fd phagemid vectors are also used, since they can be easily switched from the display of functional Ig-like fragments on the surface of Fd-bacteriophage to secreting soluble Ig-like fragments in E. coli. Phage-displayed recombinant protein fusions with the N-terminus of the gene III coat protein are made possible by an amber codon strategically positioned between the two protein genes. In amber suppressor strains of E. coli, the resulting Ig domain-gene III fusions become anchored in the phage coat.

A selection process based on protein affinity can be applied to any high-affinity binding reagents such as antibodies, antigens, receptors and ligands (see, for example, Winter and Milstein, (1991) Nature 349:293-299, the entire contents of which are incorporated herein by reference). Thus, the selection of the highest affinity binding protein displayed on bacteriophage is coupled to the recovery of the gene encoding that protein. Ig-displaying phage can be affinity selected by binding to cognate binding partners covalently coupled to beads or adsorbed to plastic surfaces in a manner similar to ELISA or solid phase radioimmunoassays. While almost any plastic surface will adsorb protein antigens, some commercial products are especially formulated for this purpose, such as Nunc Immunotubes.

Ribosomal display libraries involve polypeptides synthesized de novo in cell-free translation systems and displayed on the surface of ribosomes for selection purposes (Hanes and Pluckthun, (1997) Proc Natl Acad Sci USA 94:4937-4942; He and Taussig, (1997) Nucl Acids Res 25:5132-5134). The “cell-free translation system” comprises ribosomes, soluble enzymes required for protein synthesis (usually from the same cell as the ribosomes), transfer RNAs, adenosine triphosphate, guanosine triphosphate, a ribonucleoside triphosphate regenerating system (such as phosphoenol pyruvate and pyruvate kinase), and the salts and buffer required to synthesize a protein encoded by an exogenous mRNA. The translation of polypeptides can be made to occur under conditions which maintain intact polysomes, i.e. where ribosomes, mRNA molecule and translated polypeptides are associated in a single complex. This effectively leads to “ribosome display” of the translated polypeptide.

For selection, the translated polypeptides, in association with the corresponding ribosome complex, are mixed with a target molecule which is bound to a matrix (e.g. Dynabeads). The target molecule may be any compound of interest (or a portion thereof) such as a DNA molecule, a protein, a receptor, a cell surface molecule, a metabolite, an antibody, a hormone or a virus. The ribosomes displaying the translated polypeptides will bind the target molecule and these complexes can be selected and the mRNA re-amplified using RT-PCR.

Although there are several alternative approaches to modify binding molecules, the general approach for all displayed proteins conforms to a pattern in which individual binding reagents are selected from display libraries by affinity to their cognate receptor. The genes encoding these reagents are modified by any one or combination of a number of in vivo and in vitro mutation strategies and constructed as a new gene pool for display and selection of the highest affinity binding molecules.

### B7-1 (CD80) Protein and B7-2 (CD86) Protein

The B7 protein is a peripheral membrane protein found on activated antigen presenting cells (APC) that, when paired with either a CD28 or CD152 (CTLA-4) surface protein on a T-cell, can produce a co-stimulatory signal or a co-inhibitory signal to enhance or decrease the activity of aN MHC-TCR signal between the APC and the T cell, respectively. As well as being present on activated APCs, B7 is also found on T-cells.

The B7 protein comprises a number of family members which include B7-1, B7-2, B7-DC, B7-H1 to B7-H7. The B7-1 protein is also referred to as CD80 and binds to CD28 and CTLA-4 (cytotoxic T-lymphocyte-associated protein 4). The UniProt reference of the human sequence of B7-1 (CD80) is P33681.

In one example, the CTLA-4 binding domain binds to B7-1 human protein. In one example the CTLA-4 binding domain binds to the B7-2 protein.

### Sclerostin

Sclerostin is a secreted glycoprotein with a C-terminal cysteine knot-like domain and sequence similarity to the DAN (differential screening-selected gene aberrative in neuroblastoma) family of bone morphogenic protein (BMP) antagonists. Sclerostin is produced by the osteocyte and has anti-anaebolic effects of bone formation. The UniProt reference of the human sequence is Q9BQB4.

In one example, the CTLA-4 binding domain binds to sclerostin human protein.

### Measuring Binding Affinity

Binding of epitopes can be measured by conventional antigen binding assays, such as ELISA, by fluorescence based techniques, including FRET, or by techniques such as surface plasmon resonance which measure the mass of molecules. Specific binding of a CTLA-4 binding domain to an antigen or epitope can be determined by suitable assay, including, for example, Scatchard analysis and/or competitive binding assays such as radioimmunoassay (RIA), enzyme immunoassays such as ELISA and sandwich competition assays.

Competition assays such as surface plasmon resonance assays can be used to determine whether a CTLA-4 binding domain which has been engineered to bind a particular target is capable of doing so. By way of illustration, a CTLA-4 binding domain can be engineered to bind to the stem cell factor receptor (CSFR or c-kit receptor) and tested for its ability to compete with binding of the natural ligand (c-kit). In vitro competition assays for determining the ability of a CTLA-4 binding domain to compete for binding to a target as well as determining the dissociation constant (KD) are known in the art.

The binding affinity or dissociation constant (KD) of the interaction between the CTLA-4 binding domain and its respective target can be measured by a number of methods known in the art. Such methods include, but are not limited to, fluorescence titration, competition ELISA, calorimetric methods, such as isothermal titration calorimetry (ITC) and surface plasmon resonance (BIAcore) or Bio-layer interferometry (e.g. Blitz system (ForteBio)).

A preferred surface plasmon resonance assay is BIAcore which is known in the art.

Most binding moieties have KD values in the low micromolar (10-6) to nanomolar (10-7 to 10-9) range. High affinity binding moieties are generally considered to be in the low nanomolar range (10-9) with very high affinity binding moieties being in the picomolar (10-12) range.

The complex formation between the respective moiety and its target is influenced by many different factors such as the concentrations of the respective binding partners, the presence of competitors. pH and the ionic strength of the buffer system used, and the experimental method used for determination of the KD (for example, fluorescence titration, competition ELISA or surface plasmon resonance) or even the mathematical algorithm which is used for evaluation of the experimental data.

It is therefore clear to the person skilled in the art that the KD values may vary within a certain experimental range, depending on the method and experimental setup that is used for determining the affinity of a particular CTLA-4 binding domain for a given target. This means that there may be a slight deviation in the measured KD values or a tolerance range depending on whether the KD value was determined by surface plasmon resonance (Biacore), by competition ELISA or by “direct ELISA”.

In a preferred example, the KD value is determined by using surface plasmon resonance assays, e.g., using BIAcore surface plasmon resonance (Cytiva Life Sciences, Marlborough, MA, USA) to an immobilised target.

Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of the protein or the BDM for unrelated amino acid sequences. Affinity of a protein or BDM to a target (e.g. protein antigen) can be, for example, from about 100 nanomolar (nM) to about 0.1 nM, from about 100 nM to about 1 picomolar (pM), or from about 100 nM to about 1 femtomolar (fM) or more.

In one example, the protein has an affinity measured by KD of about 200 nM or lower, about 100 nM or lower, about 50 nM or lower, about 25 nM or lower, about 10 nM or lower, of about 5 nM or lower, of about 1 nM or lower or of about 0.5 nM or lower.

In one example, the CTLA-4 binding domain has an affinity measured by KD of about 200 nM or lower, about 100 nM or lower, about 50 nM or lower, about 25 nM or lower, 10 nM or lower, of about 5 nM or lower, of about 1 nM or lower or of about 0.5 nM or lower.

Bio-layer interferometry is a label-free technology for measuring biomolecular interactions within the interactome. It is an optical analytical technique that analyses the interference pattern of white light reflected from two surfaces: a layer of immobilized protein on the biosensor tip, and an internal reference layer. Any change in the number of molecules bound to the biosensor tip causes a shift in the interference pattern that can be measured in real-time.

The binding between a ligand immobilized on the biosensor tip surface and an analyte in solution produces an increase in optical thickness at the biosensor tip, which results in a wavelength shift, ΔA which is a direct measure of the change in thickness of the biological layer. Interactions are measured in real time, providing the ability to monitor binding specificity, rates of association and dissociation, or concentration, with precision and accuracy.

Only molecules binding to or dissociating from the biosensor can shift the interference pattern and generate a response profile. Unbound molecules, changes in the refractive index of the surrounding medium, or changes in flow rate do not affect the interference pattern. This is a unique characteristic of bio-layer interferometry and extends its capability to perform in crude samples used in applications for protein-protein interactions, quantitation, affinity, and kinetics.

### Target Molecules

The target molecule according to the present disclosure is preferably an antigen. The antigen may be selected from a protein, a glycan, a lipid, a lipoprotein or nucleic acid. The protein may be a soluble protein or membrane bound protein. Examples of soluble proteins include, but are not limited to transcription factors, antibodies, growth factors, blood proteins (e.g. albumin), or drugs (e.g. steroid, pharmaceutical drugs etc.). Types of membrane bound proteins include growth factor receptors, tumour markers, or markers which mediate transport into a cell (e.g. transferrin), or Fc receptor.

The nucleic acid target may be DNA, RNA or a combination of DNA and RNA.

The target antigen may be a tumour associated antigen. Such tumour associated antigens include, but are not limited to, MUC-1 and peptide fragments thereof, protein MZ2-E, polymorphic epithelial mucin, folate-binding protein LK26, MAGE-1 or MAGE-3 and peptide fragments thereof, Human chorionic gonadotropin (HCG) and peptide fragments thereof, Carcinoembryonic antigen (CEA) and peptide fragments thereof, Alpha fetoprotein (AFP) and peptide fragments thereof, Pancreatic oncofetal antigen and peptide fragments thereof, CA 125, 15-3, 19-9, 549, 195 and peptide fragments thereof, Prostate-specific antigens (PSA) and peptide fragments thereof, Prostate-specific membrane antigen (PSMA) and peptide fragments thereof, Squamous cell carcinoma antigen (SCCA) and peptide fragments thereof, Ovarian cancer antigen (OCA) and peptide fragments thereof, Pancreas cancer associated antigen (PaA) and peptide fragments thereof, Her1/neu and peptide fragments thereof, gp-100 and peptide fragments thereof, mutant K-ras proteins and peptide fragments thereof, mutant p53 and peptide fragments thereof, nonmutant p53 and peptide fragments thereof, truncated epidermal growth factor receptor (EGFR), chimeric protein p210BCR-ABL, telomerase and peptide fragments thereof, survivin and peptide fragments thereof, Melan-A/MART-1 protein and peptide fragments thereof, WT1 protein and peptide fragments, LMP2 protein and peptide fragments, HPV E6 E7 protein and peptide fragments, Idiotype protein and peptide fragments, NY-ESO-1 protein and peptide fragments, PAP protein and peptide fragments, cancer testis proteins and peptide fragments, and 5T4 protein and peptide fragments.

The target antigen may be an antigen or epitope present on a cell located within the heart, blood system, lungs, intestine, stomach, rectum, prostate, thyroid, liver or oesophagus.

### Expression Vectors

An “expression vector” as used herein encompasses a vector, e.g. circular or linear, single- or double-stranded, natural or engineered extrachromosomal plasmid vectors, cosmids, viral vectors, expression vectors, gene transfer vectors, minicircle vectors, and artificial chromosomes, and the like, suitable for expressing a polynucleotide which encodes a CTLA-4 binding domain.

In one example, the expression vector is a plasmid display vector.

In another example, the expression vector is a minicircle DNA vector. A “minicircle DNA vector” may be referred to as “minicircle vector” or “minicircle” and is a small (usually in the range of 3-4 kb, approximately 3-4 kb or usually no larger than 10 kb) circular, episomal plasmid derivative wherein all prokaryotic vector parts (e.g., bacterial origin of replication, genes associated with bacterial propagation of plasmids) have been removed. Since minicircle vectors contain no prokaryotic DNA sequences, they are less likely to be perceived as foreign and destroyed when they are employed as vehicles for transferring transgenes into mammalian cells.

The use of a minicircle DNA vector to carry and transfer the transgene expression cassette allows mammalian cells to be transfected (e.g., directly) without utilizing an intermediate eukaryotic host system (e.g., insect cell line production system). Furthermore, the size of minicircle vectors (which are smaller than standard plasmid vectors) and the lack of extraneous bacterial sequences enhance transfection of cells and enable an extended duration of transgene expression within the mammalian host cell. For example, a minicircle vector is smaller than a standard vector as it lacks extraneous bacterial sequences found on plasmids. Differences in size between plasmid vectors and minicircle vectors can be attributed to the lack of extraneous bacterial sequences, inclusion of an insubstantial amount of extraneous bacterial sequences in comparison to the overall size of the vector, such as appreciably smaller in comparison to the plasmid, and variations thereof. Prolonged high levels of transgene expression by minicircles in mammalian hosts can also be facilitated by in the incorporation of strong and constitutive promoters such as SV40, CMV, UBC, EF1A, PGK and CAGG.

Suitable minicircle vectors are described, for example in Mun et al (2016) Biomaterials 101 (2016) 310-320; and Gaspar et al (2014) Expert Opin. Biol. Ther. 15 (3): 1-27.

To produce the chimeric binding constructs of the present disclosure, a nucleic acid sequence encoding a CTLA-4 binding domain sequence as described herein is cloned by standard methods into a suitable expression vector.

Expression vectors encoding the CTLA-4 binding domains can integrate into the genome of the mammalian cell and replicate as the host genome replicates. Alternatively, expression vectors encoding the CTLA-4 binding domains can contain origins of replication allowing for extrachromosomal replication.

Expression vector components may also include, for example, one or more of the following: an enhancer element, a promoter, polyadenylation sequences and a transcription termination sequence.

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells sub-cloned for growth in suspension culture; baby hamster kidney cells (BHK, ATCC CCL 10); or Chinese hamster ovary cells (CHO).

Means for introducing expression vectors into a mammalian cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, retroviral transduction, electroporation and microparticle bombardment such as by using DNA-coated tungsten or gold particles (Agracetus Inc., WI, USA) amongst others.

### Leader Sequences

In one example, the leader sequence peptide is a sequence of 16 to 20 amino acids at the N-terminus of some eukaryotic proteins that determines their ultimate destination. Proteins that are made and function in the cytosol lack leader sequences. Proteins destined for specific organelles require signal sequences appropriate for each organelle. The leader sequence for a protein destined to enter the endoplasmic reticulum may contain hydrophobic amino acids that become embedded in the lipid bilayer membrane, and it functions to guide the nascent protein to a receptor protein that marks the position of a pore in the membrane. Once the protein passes into the cisternal lumen through the pore, the leader segment may be cleaved from the protein. For example, the leader sequence peptide of an interferon protein allows the cell to secrete the interferon, but is removed from the mature molecule during the secretion process. The leader sequence peptide is also often referred to as the signal peptide.

Examples of suitable leader sequences include the following:

| Leader sequence Name | Sequence |
|---|---|
| Mouse Ig Kappa | METDTLLLWVLLLWVPGSTGD (SEQ ID NO: 16) |
| Human OSM | MGVLLTQRTLLSLVLALLFPSMASM (SEQ ID |
| | NO: 17) |
| VSV-G | MKCLLYLAFLFIGVNC (SEQ ID NO: 18) |
| Human IgG2 H | MGWSCIILFLVATATGVHS (SEQ ID NO: 19) |
| BM40 | MRAWIFFLLCLAGRALA (SEQ ID NO: 20) |
| Secrecon | MWWRLWWLLLLLLLLWPMVWA (SEQ ID |
| | NO: 21) |
| Human IgKVIII | MDMRVPAQLLGLLLLWLRGARC (SEQ ID |
| | NO: 22) |
| Human CD33 | MPLLLLLPLLWAGALA (SEQ ID NO: 23) |
| tPA | MDAMKRGLCCVLLLCGAVFVSPS (SEQ ID |
| | NO: 24) |
| Human Chymotrypsinogen | MAFLWLLSCWALLGTTFG (SEQ ID NO: 25) |
| Human trypsinogen-2 | MNLLLILTFVAAAVA (SEQ ID NO: 26) |
| Human IL-2 | MYRMQLLSCIALSLALVTNS (SEQ ID NO: 27) |
| Gaussia luc | MGVKVLFALICIAVAEA (SEQ ID NO: 28) |
| Albumin(HSA) | MKWVTFISLLFSSAYS (SEQ ID NO: 29) |
| Influenza Haemagglutinin | MKTIIALSYIFCLVLG (SEQ ID NO: 30) |
| Human insulin | MALWMRLLPLLALLALWGPDPAAA (SEQ ID |
| | NO: 31) |
| Silkworm Fibroin LC | MKPIFLVLLVVTSAYA (SEQ ID NO: 32) |

### Transmembrane Domain

The term “transmembrane domain” refers to a polypeptide or protein which is encoded by a nucleic acid sequence and which comprises an optional extracellular part, a transmembrane domain and an optional cytosolic tail. A transmembrane domain is any three-dimensional protein structure which is thermodynamically stable in a membrane and usually comprises a single transmembrane alpha helix of a transmembrane protein, predominantly composed of hydrophobic amino acids. The length of the transmembrane domain is in average 21 amino acids, but might vary between 4 to 48 amino acids. A transmembrane domain comprises an optional N-terminal extracellular connecting stretch of amino acids and a transmembrane domain. In some embodiments, the transmembrane domain may further comprise a C-terminal cytoplasmic amino acid stretch or an intracellular domain.

Transmembrane domains of use include, but are not limited to, the transmembrane domain of the human platelet-derived growth factor receptor (PDGFR) gene (Swissprot entry P16234), human asialoglycoprotein receptor (Swissprot entry P07306), human and murine B7-1 (human: Swissprot entry P33681 and murine: Swissprot entry Q00609), human ICAM-1 (Swissprot entry P05362), human erbb1 (Swissprot entry P00533), human erbb2 (Swissprot entry P04626), human erbb3 (Swissprot entry P21860), human erbb4 (Swissprot entry Q15303), human fibroblast growth factor receptors such as FGFR 1 (Swissprot entry P11362), FGFR2 (Swissprot entry P21802), FGFR3 (Swissprot entry P22607), FGFR4 (Swissprot entry P22455), human VEGFR-1 (Swissprot entry P17948), human VEGFR-2 (Swissprot entry P35968), human erythropoietin receptor (Swissprot entry P19235), human PRL-R, prolactin receptor (Swissprot entry P16471), human EphA1, Ephrin type-A receptor 1 (Swissprot entry P21709), human insulin (Swissprot entry P06213), Insulin-like growth factor 1 receptor (IGFR1, Swissprot entry P08069, SEQ ID NO: 181), human receptor-like protein tyrosine phosphatases (Swissprot entries Q12913, P23471, P23467, P18433, P23470, P23469, P23468), human neuropilin (Swissprot entry P014786), human major histocompatibility complex class II (alpha and beta chains), human integrins (alpha and beta families), human Syndecans, human Myelin protein, human cadherins, human synaptobrevin-2 (Swissprot entry P63027), human glycophorin-A (GpA, Swissprot entry P02724, SEQ ID NO: 185), human Bnip3 (Swissprot entry Q12983), human APP (Swissprot entry P05067), amyloid precursor protein (Swissprot entry PODJI8), human T-cell receptor alpha gene (PTCRA, Swissprot entry PQ6ISU1) and T-cell receptor beta, CD3 gamma (Swissprot entry P09693), CD3 delta (Swissprot entry P04234), CD3 zeta (Swissprot entry P20963), and CD3 epsilon (CD3E, Swissprot entry P07766), human Serine/threonine-protein kinase receptor R3 (ACVL1, Swissprot entry P37023), human Anthrax toxin receptor 2 (ANTR2, Swissprot entry P58335), human T-cell surface glycoprotein CD4 (CD4, Swissprot entry P01730), human Receptor-type tyrosine-protein phosphatase mu (PTPRM, Swissprot entry P28827, SEQ ID NO: 177), human Tumor necrosis factor receptor superfamily member 5 (TNR5, Swissprot entry P25942). Human Integrin beta-1 (ITB1, Swissprot entry P05556), human HLA class I histocompatibility antigen, B-7 alpha chain (Swissprot entry P01889), human Thrombomodulin (TRBM, Swissprot entry P07204), human Interleukin-4 receptor subunit alpha (IL4RA, Swissprot entry P24394), human Low-density lipoprotein receptor-related protein 6 (LRP6, Swissprot entry 075581), human High affinity immunoglobulin epsilon receptor subunit alpha (FCERA, Swissprot entry P12319), human Killer cell immunoglobulin-like receptor 2DL2 (K12L2, Swissprot entry P43627), human Cytokine receptor common subunit beta (IL3RB, Swissprot entry P32927), human Integrin alpha-IIb (ITA2B, Swissprot entry P08514), human T-cell-specific surface glycoprotein CD28 (CD28, Swissprot entry P10747).

An immunoglobulin transmembrane domain may also be employed. Suitable examples include the transmembrane domain from the human immunoglobulin genes IGHA1 (NCBI access code: M60193), IGHA2 (NCBI access code: M60194), IGHD (NCBI access code: K02881), IGHE (NCBI access code: X63693), IGHG1 (NCBI access code: X52847), IGHG2 (NCBI access code: AB006775), IGHG3 (NCBI access code: D78345), IGHG4 (NCBI access code: AL928742), IGHGP (NCBI access code: X52849), IGHM (NCBI access code: X14940) as well as the transmembrane domains from the murine immunoglobulin genes IGHA1 (NCBI access code: K00691), IGHD (NCBI access code: J00450), IGHE (NCBI access code: X03624, U08933), IGHG1 (NCBI access code: J00454, J00455), IGHG2A (NCBI access code: J00471), IGHG2B (NCBI access code: J00462, D78344), IGHG3 (NCBI access code: X00915, V01526), IGHM (NCBI access code: J00444).

In one example the transmembrane domain used is selected form the group consisting of the PDGFR transmembrane domain, human B7-1 transmembrane domain, the murine B7-1 transmembrane domain, the human asialoglycoprotein receptor transmembrane domain and the erbb-2 transmembrane domain.

In one example the transmembrane domain is the PDGFR transmembrane domain.

In another example the transmembrane domain is the wild type CTLA-4 transmembrane domain or a variant thereof. For example, the CTLA-4 binding domain can be attached to its naturally occurring transmembrane domain wherein the residues that confer dimer formation have been deleted.

### Linkers

The linker can facilitate enhanced flexibility, and/or reduce steric hindrance between any two proteins. The linker can be of natural origin, such as a sequence determined to exist in random coil between two domains of a protein. An exemplary linker sequence is the linker found between the C-terminal and N-terminal domains of the RNA polymerase alpha subunit. Other examples of naturally occurring linkers include linkers found in the 1CI and LexA proteins.

Within the linker, the amino acid sequence may be varied based on the preferred characteristics of the linker as determined empirically or as revealed by modelling. Considerations in choosing a linker include flexibility of the linker, charge of the linker, and presence of some amino acids of the linker in the naturally-occurring subunits. The linker can also be designed such that residues in the linker contact DNA, thereby influencing binding affinity or specificity, or to interact with other proteins. In some cases, particularly when it is necessary to span a longer distance between subunits or when the domains must be held in a particular configuration, the linker may optionally contain an additional folded domain.

In some examples it is preferable that the design of a linker involve an arrangement of domains which requires the linker to span a relatively short distance, preferably less than about 10 Angstroms (Å). However, in certain embodiments, linkers span a distance of up to about 50 Å or more.

The term ‘peptide linker’ refers to a short peptide fragment that connects or couples the CTLA-4 binding domain to the transmembrane domain. The linker is preferably made up of amino acids linked together by peptide bonds. For example, the peptide linker can comprise small amino acid residues or hydrophilic amino acid residues (e.g. glycine, serine, threonine, proline, aspartic acid, asparagine, etc). For example, the peptide linkers are peptides with an amino acid sequence with a length of at least 5 amino acids, or with a length of about 5 to about 100 amino acids, or with a length of about 10 to 50 amino acids, or a length of about 10 to 15 amino acids.

In one example, the linker is made up of a majority of amino acids that are sterically unhindered such as glycine and alanine. Thus in a further example, the linkers are polyglycines, polyalanines or polyserines.

One skilled in the art would appreciate that many commonly used peptide linkers may be used in embodiments of the present disclosure. In certain embodiments, the short peptide linkers may comprise repeat units to increase the linker length. For example, a double, triple or quadruple repeated linker. In one example, the linker comprises a formula (Gly-Gly-Gly-Gly-Ser) n or comprising the formula (Ser-Gly-Gly-Gly-Gly) n Ser wherein n is a number from 3 to 6.

In one example, the linker comprises or consist of the sequence SGGGGSGGGGSGGGGS (SEQ ID NO:14) or SGGGGSGGGGSGGGGSGGGGS (SEQ ID NO: 15).

Non-peptide linkers are also possible. For example, alkyl linkers such as —NH—(CH2)s-C(O)—, wherein s=2-20 could be used. These alkyl linkers may be further substituted by any non-sterically hindering group such as lower alkyl (e.g. C1-C6), lower acyl, halogen (e.g. Cl, Br), CN, NH2, phenyl. An exemplary non-peptide linker is a PEG linker having a molecular weight of 100 to 5000 kD, preferably 100 to 500 kD.

Examples of other linkers which are suitable for use include GSTVAAPS, TVAAPSGS or GSTVAAPSGS or multiples of such linkers. Other examples include (TVSDVP)n (GS)m wherein n=1 and m=1 or wherein n=2 and m=1 or wherein n=2 and m=0.

In another example, the linker is GS.

### Mesenchymal Lineage Precursor or Stem Cells

As used herein, the term “mesenchymal lineage precursor or stem cells” (MLPCs) refers to undifferentiated multipotent cells that have the capacity to self-renew while maintaining multipotency and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells.

The term “mesenchymal lineage precursor or stem cells” includes both parent cells and their undifferentiated progeny. The term also includes mesenchymal precursor cells (MPC), multipotent stromal cells, mesenchymal stem cells (MSCs), perivascular mesenchymal lineage precursor or stem cells, and their undifferentiated progeny. Accordingly, in an example, the mesenchymal lineage precursor or stem cells are mesenchymal stem cells.

Mesenchymal lineage precursor or stem cells can be autologous, allogeneic, xenogeneic, syngeneic or isogeneic. Autologous cells are isolated from the same individual to which they will be reimplanted. Allogeneic cells are isolated from a donor of the same species. Xenogeneic cells are isolated from a donor of another species. Syngeneic or isogeneic cells are isolated from genetically identical organisms, such as twins, clones, or highly inbred research animal models.

In an example, the mesenchymal lineage precursor or stem cells are allogeneic. In an example, the allogeneic mesenchymal lineage precursor or stem cells are culture expanded and cryopreserved.

In an example, mesenchymal lineage precursor or stem cells express STRO-1 and one or more integrins. Integrins are a class of cell adhesion receptors that mediate both cell-cell and cell-extracellular matrix adhesion events.

In an example, mesenchymal lineage precursor or stem cells express STRO-1 and coxsackievirus and adenovirus receptor. In another example, mesenchymal lineage precursor or stem cells express STRO-1, coxsackievirus and adenovirus receptor and one or more of the above referenced integrin's.

In another example, the mesenchymal lineage precursor or stem cells are CD29+, CD54+, CD73+, CD90+, CD102+, CD105+, CD106+, CD166+, MHC1+ MSCs.

In one example, the mesenchymal lineage precursor or stem cells are MSCs. The MSCs may be a homogeneous composition or may be a mixed cell population enriched in MSCs. Homogeneous MSC compositions may be obtained by culturing adherent bone marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. MSC prepared by conventional plastic adherence isolation relies on the non-specific plastic adherent properties of CFU-F.

The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for mesenchymal lineage precursor or stem cells comprises at least about 0.1% or 0.5% or 1% or 2% or 5% or 10% or 15% or 20% or 25% or 30% or 50% or 75% mesenchymal lineage precursor or stem cells.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, as described and/or exemplified herein, cells (e.g., mesenchymal precursor cells) expressing STRO-2 and/or STRO-3 (TNAP) and/or STRO-4 and/or VCAM-1 and/or CD146 and/or 3G5 also express STRO-1 (and can be STRO-1bright). Accordingly, an indication that cells are STRO-1+ does not mean that the cells are selected solely by STRO-1 expression. In one example, the cells are selected based on at least STRO-3 expression, e.g., they are STRO-3+ (TNAP+). For example, the MPCs can be isolated from bone mononuclear cells with an anti-STRO-3 antibody.

Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+ pericytes) or any one or more of the tissues recited herein.

In one example, the cells used in the present disclosure express one or more markers individually or collectively selected from the group consisting of TNAP+, VCAM-1+, THY-1+, STRO-2+, STRO-4+ (HSP-90B), CD45+, CD146+, 3G5+ or any combination thereof.

In a preferred embodiment of the present disclosure, the mesenchymal lineage precursor or stem cells are obtained from a master cell bank derived from mesenchymal lineage precursor or stem cells enriched from the bone marrow of healthy volunteers. The use of mesenchymal lineage precursor or stem cells derived from such a source is particularly advantageous for subjects who do not have an appropriate family member available who can serve as the mesenchymal lineage precursor or stem cell donor, or are in need of immediate treatment and are at high risk of relapse, disease-related decline or death, during the time it takes to generate mesenchymal lineage precursor or stem cells.

Mesenchymal lineage precursor or stem cells encompassed by the present disclosure may also be cryopreserved prior to administration to a subject. In an example, mesenchymal lineage precursor or stem cells are culture expanded and cryopreserved prior to administration to a subject.

In an example, the present disclosure encompasses mesenchymal lineage precursor or stem cells as well as progeny thereof, soluble factors derived therefrom, and/or extracellular vesicles isolated therefrom. In another example, the present disclosure encompasses mesenchymal lineage precursor or stem cells as well as extracellular vesicles isolated therefrom. For example, it is possible to culture expand mesenchymal precursor lineage or stem cells of the disclosure for a period of time and under conditions suitable for secretion of extracellular vesicles into the cell culture medium. Secreted extracellular vesicles can subsequently be obtained from the culture medium for use in therapy.

The term “extracellular vesicles” as used herein, refers to lipid particles naturally released from cells and ranging in size from about 30 nm to as a large as 10 microns, although typically they are less than 200 nm in size. They can contain proteins, nucleic acids, lipids, metabolites, or organelles from the releasing cells (e.g., mesenchymal stem cells; STRO-1+ cells).

The term “exosomes” as used herein, refers to a type of extracellular vesicle generally ranging in size from about 30 nm to about 150 nm and originating in the endosomal compartment of mammalian cells from which they are trafficked to the cell membrane and released. They may contain nucleic acids (e.g., RNA; microRNAs), proteins, lipids, and metabolites and function in intercellular communication by being secreted from one cell and taken up by other cells to deliver their cargo.

### Pluripotent Stem Cells

As used herein, the expression “pluripotent stem cell” refers to a cell capable of differentiating into all three germ layers (i.e. endoderm, ectoderm and mesoderm). According to some embodiments of the invention, the expression “pluripotent stem cells” includes embryonic stem cells (ESCs) and induced pluripotent stem cells (iPS cells).

The expression “embryonic stem cells” are cells obtained from embryonic tissues formed after pregnancy (e.g. blastocysts) (pre-implantation (ie, pre-implantation blastocysts)), late-implantation/early protozoal formation. Expanded blastocyst cells (EBC) obtained from blastocysts (see WO 2006/04763), and obtained from fetal genital tissue at any time during pregnancy, preferably 10 weeks before pregnancy.

According to some embodiments of the invention, the pluripotent stem cells of the invention are, for example, embryonic stem cells derived from humans or primates (eg monkeys).

It will be appreciated that commercially available stem cells can also be used in this aspect of the invention. Human ES cells can be purchased from the NIH human embryonic stem cells registry (www.escr.nih.gov). Non-limiting examples of commercially available embryonic stem cell lines include BG01, BG02, BG03, BG04, CY12, CY30, CY92, CY10, TE03, TE04 and TE06.

As used herein, the expression “induced pluripotent stem (iPS) cell” (or embryonic pluripotent stem cell) refers to proliferative and pluripotent stem cells obtained by dedifferentiation of somatic cells (e.g. adult somatic cells).

According to some embodiments of the invention, iPS cells are characterized by proliferative potential similar to that of ESCs and can therefore be maintained and proliferated in culture for almost infinite time.

IPS cells can be given pluripotency by genetic engineering to reprogram the cells and obtain embryonic stem cell characteristics. They can be produced from somatic cells by inducing expression of −4, Sox2, Kfl4 and c-Myc. In addition, or instead, the iPS cells of the invention are derived from somatic cells by inducing expression of Oct4, Sox2, Nanog and Lin28, essentially as described in Yu et al., (2007) Science 318 (5858): 1917-1920 and Nakagawa et al., (2008) Science 322 (5903): 949-953. It should be noted that genetic manipulation (reprogramming) of somatic cells can be performed using any known method, such as the use of plasmids or viral vectors, or by induction without any integration into the genome (Yu J. et al., Science. 2009, 324:797-801).

The iPS cells of the present invention may be embryonic fibroblasts, fibroblasts formed from hESC, fetal fibroblasts, encapsulative fibroblasts, adult skin and skin tissue. They are available by inducing dedifferentiation of lymphocytes, as well as adult liver and gastric cells.

IPS cell lines are also available via cell banks such as the WiCell bank. As non-limiting examples of commercially available iPS cell lines, iPS foreskin clone 1 [WiCell catalog number: iPS (foreskin)-1-DL-1], iPSIMR90 clone 1 [WiCell catalog number: iPS (IMR90)-1-DL-1]], And iPSIMR90 clone 4 [WiCell catalog number: iPS (IMR90)-4-DL-1].

According to some embodiments of the present invention, the induced pluripotent stem cells are human induced pluripotent stem cells.

### Therapeutic Agents

In one embodiment the mammalian cell has been modified to carry a therapeutic agent to a target cell or tissue.

In an example the therapeutic agent is a recombinant virus. The term “recombinant virus” is used in the context of the present disclosure to refer to viruses that express a transgene of interest in a cell (or population thereof) defined herein. In an example, a recombinant virus expresses a transgene that can kill cancer cells. In an example, the recombinant virus comprises a herpes simplex virus backbone. In an example, the recombinant virus is a herpes simplex virus.

In an example, the recombinant virus expresses a gene which enhances the immune response against an infected tumour cell. For example, the gene(s) may be GM-CSF, FLT3L, CCL3, CCL5, IL2, IL4, IL6, IL12, IL15, IL 18, IFNA1, IFNB1, IFNG, CD80, 4-1BBL, CD40L, a heatshock protein (HSP) or a combination thereof.

In one example the therapeutic agent is an oncolytic virus. The term “oncolytic virus” is used in the context of the present disclosure to refer to viruses that are able to infect and reduce growth of cancer cells. For example, oncolytic viruses can inhibit cell proliferation. In another example, oncolytic viruses can kill cancer cells. In an example, the oncolytic virus preferentially infects and inhibits growth of cancer cells compared with corresponding normal cells. In another example, the oncolytic virus preferentially replicates in and inhibits growth of cancer cells compared with corresponding normal cells.

In an example, the oncolytic virus is able to naturally infect and reduce growth of cancer cells. Examples of such viruses include Newcastle disease virus, vesicular stomatitis, myxoma, reovirus, sindbis, measles and coxsackievirus. Oncolytic viruses able to naturally infect and reduce growth of cancer cells generally target cancer cells by exploiting the cellular aberrations that occur in these cells. For example, oncolytic viruses may exploit surface attachment receptors, activated oncogenes such as Ras, Akt, p53 and/or interferon (IFN) pathway defects.

In another example, oncolytic viruses encompassed by the present disclosure are engineered to infect and reduce growth of cancer cells. Exemplary viruses suitable for such engineering include oncolytic DNA viruses, such as adenovirus, herpes simplex virus (HSV) and Vaccinia virus; and oncolytic RNA viruses such as Lentivirus, Reovirus, Coxsackievirus, Seneca Valley Virus, Poliovirus, Measles virus, Newcastle disease virus, Vesicular stomatitis virus (VSV) and parvovirus such as rodent protoparvoviruses H-1PV. In an example, the oncolytic virus comprises a backbone of an above referenced virus. For example, the oncolytic virus can comprise a HSV backbone. In an example, the oncolytic virus is a HSV.

In an example, the oncolytic virus is replication-competent. In an example, oncolytic viruses selectively replicate in cancer cells when compared with corresponding normal cells and/or mesenchymal lineage precursor or stem cells. In an example, tumour specificity of oncolytic virus can be engineered to restrict virus replication by its dependence on transcriptional activities that are constitutively activated in cancer cells (i.e. conditional replication). In an example, the oncolytic virus is a conditionally replicative lentivirus. In another example, the oncolytic virus is a conditionally replicative adenovirus, reovirus, measles, herpes simplex virus, Newcatle disease virus or vaccinia.

In another embodiment the therapeutic agent is an immune response-stimulating cytokine that leads to or produces either directly or indirectly the induction, activation and/or enhancement of an immune response, preferably directed against an antigen, for example a tumour antigen. In particular, the immune response-stimulating cytokines of the invention are preferably considered as cytokines that leads to the induction, activation and/or enhancement of an immune response beneficial for the treatment of a tumour disease.

Particular kinds of cytokines may include Monokines, namely cytokines produced by mononuclear phagocytic cells, Lymphokines, namely cytokines produced by activated lymphocytes, especially Th cells, Interleukins, namely cytokines that act as mediators between leukocytes and Chemokines, namely small cytokines primarily responsible for leucocyte migration. Cytokine signalling is flexible and can induce both protective and damaging responses. They can produce cascades, or enhance or suppress production of other cytokines. Despite the various roles of cytokines, a skilled person is aware of which cytokines may be considered as immune response-stimulating and therefore applied in the treatment of a tumour disease as described herein.

Two commonly used groups of cytokines in anti-tumour therapy are the interferons and interleukins.

Interferons are cytokines produced by the immune system usually involved in an anti-viral response, but also show effectiveness in the treatment of cancer. There are three groups of interferons (IFNs): type I (IFN alpha and IFN beta), type 2 (IFN gamma) and the relatively newly discovered type III (IFN lambda). IFN alpha has been applied in the treatment of hairy-cell leukaemia, AIDS-related Kaposi's sarcoma, follicular lymphoma, chronic myeloid leukaemia and melanoma. Type I and II IFNs have been researched extensively and although both types promote the anti-tumour effects of the immune system, only type I IFNs have been shown to be clinically effective in cancer treatment so far. IFN lambda has been tested for its anti-tumour effects in animal models, and shows promise.

According to some embodiments of the present invention, the immune-response stimulatory or immune response-modulatory cytokines are preferably those involved in T cell regulation or with effector function for T cells (T cell regulatory cytokines). These cytokines exhibit desired properties with respect to inducing a pro-inflammatory microenvironment and thereby facilitating the activation of the immune system against the tumour and/or enhance the efficacy of anti-tumour immunotherapeutic treatments. Such cytokines may be able to attract immune effector cells, such as T cells, and promote the maturation of memory immune cells. Examples of these cytokines are IFN gamma, IL-2, IL-12, IL-23, IL-15 and IL-21 (refer Kelley's Textbook of Rheumatology; Firestein et al, 8th ed. (ISBN 978-1-4160-3285-4), p 367 “Cytokines”).

In another example the immune stimulatory molecule that induces T-cell proliferation and/or differentiation is CD28. CD28 (Cluster of Differentiation 28) is one of the proteins expressed on T cells that provide co-stimulatory signals, which are required for their activation. CD28 has also been found to stimulate eosinophil granulocytes, where its ligation with anti-CD28 leads to the release of IL-2, IL4, IL-13 and IFN gamma.

In another example the immune response-stimulating cytokine is a chemokine with chemotactic properties for attracting T cells, for example, CCL1, CCL2 and/or CCL17.

In another example the therapeutic agent is a checkpoint inhibitor. For example, the checkpoint inhibitor may be a PD-L1 and/or PD-1 inhibitor.

### Compositions

The mammalian cells of the present disclosure can be used as a composition when combined with a pharmaceutically acceptable carrier or excipient. Such pharmaceutical compositions are useful for administration to a subject in vivo.

Pharmaceutically acceptable carriers are physiologically acceptable to the administered patient and retain the therapeutic properties of the molecule with which it is administered. Pharmaceutically acceptable carrier and their formulations are generally described in, for example, Remington' pharmaceutical Sciences (18th ed. Ed. A Gennaro, Mack Publishing Co., Easton PA 1990). One exemplary carrier is physiological saline. The phrase “pharmaceutically acceptable carrier” as used herein means a pharmaceutically acceptable material, composition or vehicle, such as a liquid or solid filler, diluent, excipient, solvent or encapsulating material, involved in carrying or transporting the polypeptides from the administration site of one organ or portion of the body, to another organ, or portion of the body. Each carrier must be acceptable in the sense of being compatible with the other ingredients of the formulation and not injurious to the patient.

The pharmaceutically acceptable excipient may include a preservative or cryopreservative.

Pharmaceutical compositions can be formulated to be compatible with a particular route of administration, systemic or local.

Methods for preparing a molecule into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this disclosure are particularly useful for parenteral administration, such as intravenous administration or administration into a body cavity or lumen of an organ or joint. The compositions for administration will commonly comprise a solution of polypeptide dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of proteins of the present disclosure in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Non-aqueous vehicles such as mixed oils and ethyl oleate may also be used. Liposomes may also be used as carriers. The vehicles may contain minor amounts of additives that enhance isotonicity and chemical stability, e.g., buffers, preservatives or additives.

Upon formulation, cells of the present disclosure will be administered in a manner compatible with the dosage formulation and in such amount as is therapeutically/prophylactically effective.

For intravenous administration, suitable carriers include physiological saline, bacteriostatic water, Cremophor ELTM (BASF, Parsippany, N.J.) or phosphate buffered saline (PBS). The carrier can be a solvent or dispersion medium containing, for example, water, ethanol, polyol (for example, glycerol, propylene glycol, and liquid polyetheylene glycol, and the like), and suitable mixtures thereof. Antibacterial and antifungal agents include, for example, parabens, chlorobutanol, phenol, ascorbic acid and thimerosal. Isotonic agents, for example, sugars, polyalcohols such as manitol, sorbitol, and sodium chloride may be included in the composition. The resulting solutions can be packaged for use as is, or lyophilized; the lyophilized preparation can later be combined with a sterile solution prior to administration.

Compositions of the present disclosure can be combined with other therapeutic moieties or imaging/diagnostic moieties as provided herein. Therapeutic moieties and/or imaging moieties can be provided as a separate composition, or as a conjugated moiety. Linkers can be included for conjugated moieties as needed and have been described elsewhere herein.

Compositions of the present disclosure can be administered with other therapeutic agents, e.g. chemotherapeutic agents. Chemotherapeutic agents are known in the art and include cytotoxic and cytostatic drugs. Non-limiting examples include paclitaxel, cisplatin, methotrexate, doxorubicin, fludarabine etc. Other therapeutic agents are contemplated depending on the condition to be treated.

One embodiment of the present disclosure contemplates the use of any of the pharmaceutical compositions of the present disclosure to make a medicament for treating a disorder. Medicaments can be packaged in a suitable pharmaceutical package with appropriate labels wherein the label is for the indication of treating a disorder in a subject.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXAMPLES

### Methods

### Plasmid Generation

To achieve expression on the extracellular surface of mammalian cells, the DNA sequences coding for several CTLA-4 binding domains (CTLA-4 BDs) were cloned into the plasmid display vector (Invitrogen, Cat. No. V66020). The display contains the leader sequence (signal peptide) of the Immunoglobulin G (lgG) Kappa light chain, which marks the peptide for secretion, and the transmembrane domain from platelet-derived growth factor receptor beta (PDGFRB), which anchors the protein in the plasma membrane. Sequences cloned in-frame between these two features will be presented on the extracellular surface of cells transfected with the plasmid.

For the experiments described herein, the nucleic acid sequences encoding for two CTLA-4 BDs, namely BD_B7 and BD_SOST, were cloned into Display using standard techniques (restriction and ligation). BD_B7 is directed against B7.1 (CD80), and BD_SOST against human sclerostin (SOST).

The CTLA-4 sequence used in these examples comprise a C-terminal modification of the native sequence wherein the native sequence PEPCPDSDGSTG is replaced with PEPSPDSN. This sequence does not contain the C-terminus Cys residue which allows the BDM to remain in monomeric form. Both BD_B7 and BD_SOST include the addition of an amino acid A (alanine) at the C-terminus just prior to the GS linker.

### Amino Acid Sequence of BD_B7-Display Vector:

```
METDTLLLWVLLLWVPGSTGDKAMHVAQPAVVLASSRGIASFVCEYASPGKYTEVRVTVLRQ
          IgK leader BL1
ADSQVTEVCAATYMTGNELTFLDDSICTGTSSGNQVNLTIQGLRAMDTGLYICKVELMYPPPY
                            BL2 BL3
YLGIGNGTQIYVIDPEPSPDSNAGGGGSGGGGSGGGGSAEQKLISEEDLNAVGQDTQEVIVVP
                                                      GS Linker PDGFRβ TMD
HSLPFKVVVISAILALVVLTIISLIILIMLWQKKPR
Binding Loop regions underlined
TMD = transmembrane domain
Underline and italics = IgK leader sequence
Amino acid sequence of BD_SOST-Display Vector:
METDTLLLWVLLLWVPGSTGDKAMHVAQPAVVLASSRGIASFVCEYTVSWVDMEVRVTVLRQ
              IgK leader BL1
ADSQVTEVCAATYWNGRWLTFLDDSICTGTSSGNQVNLTIQGLRAMDTGLYICKVQLDPSWG
                            BL2 BL3
YYWQGYEGIGNGTQIYVIDPEPSPDSNAGGGGSGGGGSGGGGSAEQKLISEEDLNAVGQDT
                                                            GS Linker DGFRβ TMD
QEVIVVPHSLPFKVVVISAILALVVLTIISLIILIMLWQKKPR
Binding Loop regions underlined
TMD = transmembrane domain
Underline and italics = IgK leader sequence
```

### Transfection of Mammalian Cells

### Transfection of HEK293 and CHO Cells

HEK293 cells were grown in Dulbecco's modified Eagle Medium (DMEM, Gibco, Cat. No 10567014) supplemented with 10% Ultra-Low IgG Fetal Calf Serum (FCS, Gibco, Cat. No. 1921005PJ). CHO cells were grown in DMEM/F12 (Gibco, Cat. No. 10565018), supplemented with 10% Ultra-Low IgG Fetal Calf Serum (FCS, Gibco, Cat. No. 1921005PJ). The remainder of the protocol was identical for both cell types. One day before transfection, cells were plated on 96-well microtiter plates that had previously been coated with Poly-D-Lysine (PDL, Gibco, Cat. No. A38904-01) according to the manufacturer's instructions. The ideal cell density for subsequent transfection and in-cell ELISA was empirically determined to be 10,000 cells per well at time of seeding. The day after plating, cells were transfected using the Lipofectamine 3000 transfection kit (Invitrogen, Cat. No. L3000-008). The ideal amount of DNA and Lipofectamine per well was empirically determined to be 100 ng (HEK cells) or 200 ng (CHO cells) of DNA and 0.15 μl of Lipofectamine 3000. Transfections were performed according to the manufacturer's instructions.

### Transfection of Human Mesenchymal Stem Cells (MSCs)

Human mesenchymal stem cells derived from adipose tissue (aMSCs, Merck, Cat. No. SCC038, Lot No. VP1806250) and bone marrow (bMSCs, Merck, Cat. No. SCC034, Lot No. 3602371) were grown in DMEM/F12 (Gibco, Cat. No. 10565018), supplemented with 10% Ultra-Low IgG Fetal Calf Serum (FCS, Gibco, Cat. No. 1921005PJ) or in Mesenchymal Stem Cell Basal Medium (MSCBM, Lonza, Cat. No. PT-3001).

Transfection of bMSCs Via Lipofection

Since transfection of DNA, but not mock transfection, caused significant cytotoxicity, and because unspecific antibody binding is correlated with the number of cells, the transfection protocol had to be modified. One day before transfection, cells were plated on 12-well microtiter plates at a density of 50,000 cells per well at time of seeding. The day after plating, cells were transfected using the Lipofectamine Stem transfection kit (Invitrogen, Cat. No. STEM00003). The ideal amount of DNA and Lipofectamine per well was empirically determined to be 625 ng of DNA and 2.5 μl of Lipofectamine Stem per well. 30 minutes before transfection, the cells were “primed” by adding Dexamethasone at a final concentration of 100 nM and KPT-330 (Selinexor) at final concentration of 1 μM. This step greatly increases transfection efficiency and expression in MSCs (Hamann et al., Glucocorticoid Priming of Nonviral Gene Delivery to hMSCs Increases Transfection by Reducing Induced Stresses, Mol Ther Methods Clin Dev 2020) Transfections were performed according to the manufacturer's instructions. One day after transfection, cells were detached with Accutase detachment solution and counted. Cells were plated on a 96-well plate at a density of 10,000 cells per well. The in-cell ELISA was performed the following day.

In another approach to compensate for transfection-induced cytotoxicity, control cells were transfected with pDisplay DNA not containing a BDM, which caused cell death at rates comparable to transfection with BDM-containing pDisplay. In those cases, cells were plated in a 96-well plate at a density of 7,500 cells per well. The following day, cells were primed with Dexamethasone and KPT-330 as described above and then transfected using the Lipofectamine Stem transfection kit (Invitrogen, Cat. No. STEM00003). The ideal amount of DNA and Lipofectamine per well was empirically determined to be 100 ng of DNA and 0.15 μl of Lipofectamine Stem per well.

Transfection of bMSCs Via Electroporation (Nucleofection)

As an alternative to lipofection, plasmid DNA was introduced into bMSCs by electroporation using an Amaxa Nucleofector II electroporator and the Human Mesenchymal Stem Cell Nucleofector kit (Lonza, Cat. No. VVPE-1001).

Cells were grown in MSCBM in T75 flasks to approximately 90% confluence. On the day of transfection, cells in each flask were washed with 10 ml Hank's Balanced Salt Solution without Calcium and without Magnesium (HBSS^{-/-}, Gibco Cat. No. 14175095). The cells were then incubated with 3 ml 0.25% Trypsin in HBSS^{-/-} for 10-15 minutes at 37° C. and 5% CO₂. Detached cells were washed off with and resuspended in 7 ml HBSS containing Calcium and Magnesium (HBSS^{+/+}, Gibco Cat. No. 14025092). Cells were centrifuged for 10 minutes at 250×g, the supernatant was aspirated, and the pellet resuspended in 1 ml HBSS^{+/+}. Cells were counted using a hemocytometer and a volume corresponding to 1×10⁶ cells was transferred to a 1.5 ml tube. Cells were again centrifuged for 10 minutes at 250×g, the supernatant was aspirated, and the pellet resuspended in 100 μl Nucleofector solution containing 2 μg of plasmid DNA. The suspension as transferred to a nucleofection cuvette and immediately electroporated. 500 μl of pre-warmed MSCBM were added to the cells and the suspension was transferred to the well of a 6-well plate containing 1 ml of MSCBM. Cells were incubated for 15 minutes at 37° C. and 5% CO₂ before cell number and viability was assessed using a hemocytometer. An appropriate volume was then transferred to the desired vessel (e.g., for a 96-well plate 15,000 cells were added to each well; for a T25 flask up to 1×10⁶ were added).

In-Cell ELISA with Mammalian Cells

All in-cell ELISA experiments were carried out in 96-well microtiter plates. For assays in which the primary antibody was directed against the ligand of the CTLA-4 binding domain, the supernatant of cells expressing the CTLA-4 BD was aspirated, replaced with medium containing the ligand (rhSOST (R&D systems 1406-ST-025/CF) or B7-1/CD80 protein (Acro Biosystems, B71-H5259) at varying concentrations, and incubated for 1 h at RT. For assays in which binding of B-lymphocytes to cells expressing the CTLA-4 binding domain was to be assessed, cells from a lymphoblast-like cell line (Raji cells) were used. The required number of Raji cells (100,000 cells for each assay well) were centrifuged and resuspended in an appropriate volume of MSCBM (100 μl/100,000 cells), and the cell suspension was added to the wells containing bMSCs. Plates were incubated for 1 h at RT before the wells were very carefully washed 3× with D-PBS. In-cell ELISA assays were carried out two days after transfection with CTLA-4 BD plasmid DNA. The cell supernatant was aspirated, cells were washed 3× with 300 μl D-PBS (137 mM NaCl, 8.1 mM Na₂HPO₄, 2.68 mM KCl, 1.47 mM KH₂PO₄, 0.9 mM CaCl₂), 0.5 mM MgCl₂), and then fixed by adding 50 μl of a formaldehyde solution (4% in PBS, for example Santa Cruz Biotechnology, Cat. No. SANTS-281692). After 15-20 minutes of incubation at RT, the solution was aspirated and cells were washed 3× with 300 μl D-PBS-T (D-PBS containing 0.05% (w/V) Tween-20). Wells were blocked by incubating with 300 μl of 5% skim milk in D-PBS-T for 1 h at RT. The blocking buffer was aspirated and wells were washed with 3×300 μl D-PBS-T before adding 50 μl of the primary antibody diluted in D-PBS-T (see Table 1 for antibodies and their respective dilutions). After 1 h of incubation at RT the antibody solution was aspirated and wells were washed 3-5× with 300 μl D-PBS-T. A horseradish peroxidase (HRP)-conjugated secondary antibody was diluted in D-PBS-T (see Table 1), and 100 μl were added to each well. After incubation for 1 h at RT, the antibody solution was aspirated and wells were washed 3× with 300 μl D-PBS-T and 2× with 300 μl D-PBS. Then, 100 μl of HRP substrate solution (1-Step Ultra TMB, Themo Fisher, Cat. No. 34029) were added to the wells. After 5-15 minutes the reaction was stopped by adding 100 μl 1 M HCl, and absorbance at a wavelength of 450 nm was measured using a micro plate reader.

TABLE 1
Antibodies and dilutions used for in-cell ELISA
(ICE) and immunofluorecence (IF) experiments

| Antibody | Type | Dilution | Manufacturer | Cat. No. | Lot No. |
|---|---|---|---|---|---|
| Mouse Anti- | Primary/ | 1:1000 | abcam | ab110650 | GR3357159 |
| CTLA4 | monoclonal | (ICE) | | | |
| | | 1:100 (IF) | | | |
| Mouse Anti- | Primary/ | 1:1000 | Invitrogen | MA5- | WE3268024 |
| SOST | monoclonal | | | 23897 | |
| Rabbit anti- | Primary/ | 1:1000 | Abcam | ab225674 | GR3290249- |
| CD80 (B7-1) | monoclonal | | | | 2 |
| Goat anti- | Secondary/ | 1:5000 | Abcam | ab97080 | GR3391848- |
| Rabbit IgG | polyclonal | | | | 2 |
| (HRP) | | | | | |
| Donkey Anti- | Secondary/ | 1:5000 | Abcam | Ab7061 | GR3368352- |
| Mouse IgG | polyclonal | | | | 2 |
| (HRP) | | | | | |
| Goat-Anti- | Secondary/ | 1:2000 | Abcam | Ab150117 | |
| Mouse IgG | polyclonal | | | | |
| (AlexaFluor ®488) | | | | | |
| Goat-Anti- | Secondary/ | 1:2000 | Abcam | Ab150083 | |
| Rabbit IgG | polyclonal | | | | |
| (AlexaFluor ®647) | | | | | |

### Immunofluorescent Staining and Confocal Microscopy

For immunofluorescent (IF) staining and subsequent imaging, transfected bMSCs were plated on 8-well chamber slides (Nunc Lab-Tek Chamber Slide System, Permanox plastic, ThermoFisher Cat. No. 177830). One to two days after plating, the supernatant was aspirated, and wells were washed 3× with 250 μl D-PBS. Cells were fixed with 100 μl 4% formaldehyde in PBS for 15 min at RT. The formaldehyde solution was aspirated, and wells were washed 3× with D-PBS. Wells were then blocked for 1 h with 250 μl of 10% normal goat serum (NGS, ThermoFisher Cat. No. 31873) in D-PBS-T. Blocking buffer was aspirated and 100 μl of primary antibody in blocking buffer were added (see Table 1 for dilutions). After incubating for 1 h at RT, primary antibody solution was removed, and the wells were washed 3× with 250 μl D-PBS-T. 100 μl of fluorophore-labelled secondary antibody in blocking buffer (for dilutions see Table 1) were added and incubated for 1 h at RT in the dark. Wells were washed 3× with 250 μl D-PBS-T and 2× with 250 μl D-PBS. Wells were then incubated for 5 min with a 1 μg/ml 4′,6-diamidino-2-phenylindole (DAPI, Sigma Cat. No. D9543) solution in PBS to stain the nuclei. Wells were washed 3× with 250 μl D-PBS. The plastic chambers and silicone gaskets were carefully removed from the slide, and a glass cover slip was mounted using ProLong Glass Antifade mountant (ThermoFisher Cat. No. P36982). Slides were cured for 24 h at RT in the dark and then stored at 4° C. in the dark.

### Results

### Example 1 Cell Surface Expression on CTLA-4

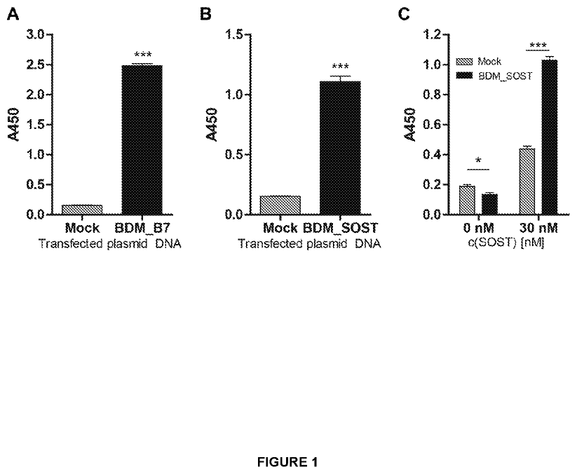

The results presented in FIG. 1 compared detection of binding domains (BDs) directed against either (A). B7-1 (CD80) or (B). Sclerostin (SOST) expressed on the cell surface of HEK293 kidney cells transfected with a BD or mock transfected with plasmid containing no BD sequence using an anti-CTLA-4 antibody. Binding of human recombinant sclerostin bound to HEK cells expressing a BD engineered to bind to sclerostin is shown in FIG. 1C.

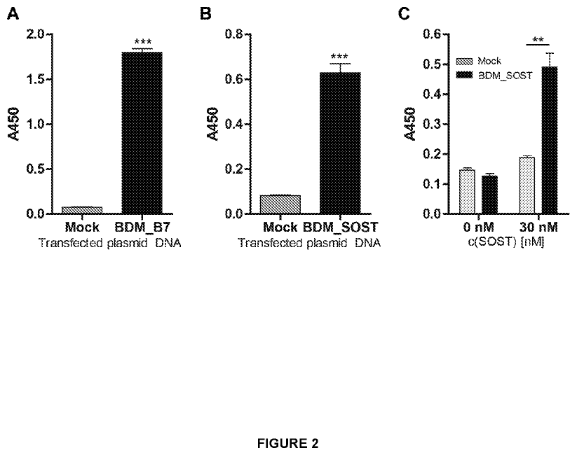

FIG. 2 reproduces the experiments of FIG. 1, however the cells that were transfected were Chinese Hamster Ovary (CHO) cells. Binding domains (BDs) directed against either (A) B7-1 (CD80) or (B) sclerostin (SOST) expressed on the cell surface of CHO cells transfected with a BD or mock transfected (empty plasmid) were detected with an anti-CTLA-4 antibody. Binding of human recombinant sclerostin bound to CHO cells expressing a BD engineered to bind to sclerostin is shown in FIG. 2C.

The inventors also examined the ability of adipose tissue-derived mesenchymal stem cells (aMSCs) to be transfected with a plasmid comprising DNA expressing a CTLA-4 binding domain (either BD_B7 or BD_SOST) or mock transfected with empty plasmid. Detection of the BDs on the cell surface was examined using an antibody specific for CTLA-4. Binding of rhSOST could not be assessed since aMSCs endogenously express SOST-binding proteins.

These results show that CTLA-4 binding domains can be expressed, functionally folded and presented on the surface membrane of a range of mammalian cells, namely HEK293, CHO, and adipose derived MSCs. The CTLA-4 binding domains presented on the surface of these cells maintain their correct configuration and are able to bind to their known binding partners.

### Example 2 MSCs Expressing CTLA-4 can Bind CD80-Expressing Cells

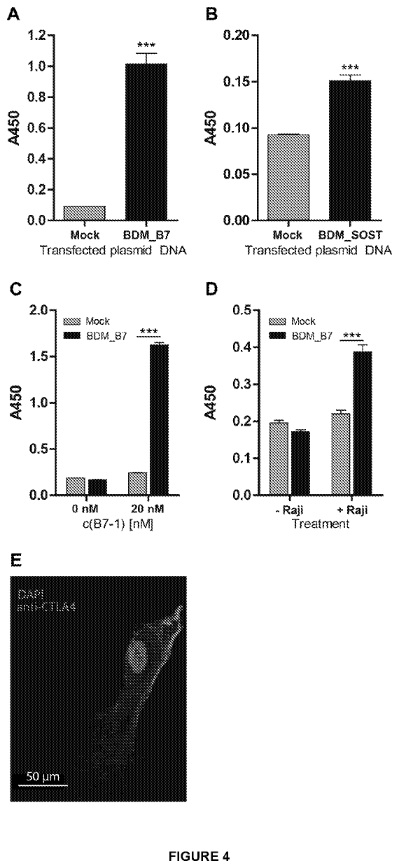

The inventors performed experiments (in cell ELISA) to show that bone marrow-derived mesenchymal stem cells (bMSCs) transfected with plasmid DNA expressing a CTLA-4 binding domain directed against B7-1 or a CTLA-4 binding domain directed against sclerostin was able to be expressed on the cell surface of bMSCs. Cell surface expression was detected using an anti-CTLA-4 specific antibody. FIG. 4A shows detection of BD_B7 and FIG. 4B shows detection of BD_SOST using an anti-CTLA-4 antibody. FIG. 4C shows detection of human B7-1 (CD80) protein bound to BD_B7 expressing bMSCs. Binding of rhSOST could not be assessed since bMSCs endogenously express SOST-binding proteins.

The BD_B7 expressing bMSCs were examined for their ability to bind to Raji cells expressing CD80. Detection of Raji cells bound to BD_B7 expressing bMSCs was made using an anti-CD80 antibody. The transfected bMSCs were stained and immunofluorescence staining observed wherein CTLA-4 BDs were located on the cell surface (stained green) using an anti-CTLA-4 antibody while nuclear staining (blue) was observed using a nuclear stain (DAPI).

These results show that CTLA-4 binding domains can be expressed, correctly folded and presented on the surface of bone marrow derived MSCs (BMSCs) and are able to bind to cells expressing an antigen to which the BD recognises.

1. A mammalian cell having a cellular membrane, where the cell is modified to express on the surface of the membrane a CTLA-4 binding domain which binds to a target molecule,

2. The mammalian cell according to claim 1 wherein:
(i) binding of the CTLA-4 binding domain to the target molecule homes the cell to the target molecule in vivo; or
(ii) binding of the CTLA-4 binding domain to the target molecule homes the target molecule to the cell in vivo.

3. (canceled)

4. The mammalian cell according to claim 1, wherein;
(i) the cell is selected from the group consisting of primate-, canine-, feline- and rodent-derived cells;
(ii) the cell belongs to anyone of the cell line families CHO, NSO, HEK293, myeloma, NOS, COS, BHK, HeLa and PER.C6;
(iii) the cell is a primary cell; or
(iv) the cell is an immune cell.

5-7. (canceled)

8. The mammalian cell according to claim 1 wherein the immune cell is selected from the group including a T cell, a cytotoxic T cell, a monocyte, a peripheral blood hematopoietic stem cell, a macrophage, an antigen presenting cell, a Natural Killer cell, a mast cell, a neutrophil, an eosinophil, a basophil, a Natural Killer T cell, a B cell, a dendritic cell, and a regulatory T cell.

9. The mammalian cell according to claim 1 wherein:
(i) the cell is a stem cell;
(ii) cell is a mesenchymal lineage precursor (MPC) or mesenchymal stem cell (MSC);
(iii) the cell is an induced pluripotent stem cell (an iPSC); or
(iv) the cell is a differentiated stem cell, MPC, or MSC.

10-14. (canceled)

15. The mammalian cell according to claim 1, wherein the CTLA-4 binding domain comprises a framework sequence having at least about 90% sequence identity to residues 1 to 25, 34 to 54, 60 to 97 and 106 to 126 of SEQ ID NO: 1, wherein SEQ ID NO: 1 consists of the sequence set forth in KAMHVAQPAVVLASSRGIASFVCEYASPGKATEVRVTVLRQADSQVTEVCAATYMTG NELTFLDDSICTGTSSGNQVNLTIQGLRAMDTGLYICKVELMYPPPYYLGIGNGTQIYVI DPEPSPDSN.

16. The mammalian cell according to claim 15, wherein the amino acid residues at positions 26 to 33, and/or positions 55 to 59 and/or positions 98 to 105 of SEQ ID NO:1 are modified or replaced with one or more heterologous sequences.

17. The mammalian cell according to claim 1, wherein the CTLA-4 binding domain comprises or consists of the sequence set forth in:
KAMHVAQPAVVLASSRGIASFVCEYXn1VRVTVLRQADSQVTEVCAATYXn2 LTFLDDSICTGTSSGNQVNLTIQGLRAMDTGLYICKVXn3LGIGNGTQIYVIDPEPSPDS N (SEQ ID NO:2) wherein X, X and X is any amino acid residue and n is a number between 5 and 15 and n1, n2, n3 indicate binding loops (BLs) 1, 2 and 3 respectively.

18. The mammalian cell according to claim 17, wherein;
(i) Xn1 is between 5 and 8 amino acids, Xn2 is between 5 and 8 amino acids, and Xn3 is between 10 and 15 amino acids; or
(ii) Xn1 is 8 amino acids and Xn2 is 5 amino acids.

19-20. (canceled)

21. The mammalian cell according to claim 1, wherein the CTLA-4 binding domain is tethered to the surface of the membrane by a transmembrane domain.

22. The mammalian cell according to claim 21, wherein the transmembrane domain is selected from the group consisting of the transmembrane domain of human platelet-derived growth factor receptor (PDGFR), human asialoglycoprotein receptor, human and murine B7-1, human ICAM-1, human erbbI, human erbb2, human erbb3, human erbb4, human fibroblast growth factor receptors such as FGFR 1, FGFR2, FGFR3, FGFR4, human VEGFR-1, human VEGFR-2, human erythropoietin receptor, human PRL-R, prolactin receptor, human EphA1, Ephrin type-A receptor 1, human insulin, IGF-1 receptors, human receptor-like protein tyrosine phosphatases, human neuropilin, human major histocompatibility complex class II (alpha and beta chains), human integrins (alpha and beta families), human Syndecans, human Myelin protein, human cadherins, human synaptobrevin-2, human glycophorin-A, human Bnip3, human APP, amyloid precursor protein, human T-cell receptor alpha and beta, CD3 gamma, CD3 delta, CD3 zeta, and CD3 epsilon.

23. (canceled)

24. The mammalian cell according to claim 1, wherein the cell has also been modified to carry a therapeutic agent, optionally wherein the therapeutic agent is an anticancer agent, an immunomodulatory agent, a recombinant virus, or a naturally occurring or modified oncolytic virus.

25.-26. (canceled)

27. The mammalian cell according to claim 1, wherein the target molecule is selected from the group consisting of a target expressed by a tumour and a target associated with the tumour stroma.

28. A method for homing mammalian cells to a target molecule in a subject, comprising administering to the subject a mammalian cell according to claim 1.

29. A chimeric binding domain comprising
(a) a leader sequence for translocating the chimeric domain across an intracellular membrane;
(b) a CTLA-4 binding domain specific for a target molecule; and
(c) a transmembrane domain which anchors the chimeric domain to the surface membrane of a mammalian cell.

30. The chimeric binding domain according to claim 29, further comprising a linker sequence located between the CTLA-4 binding domain and the transmembrane domain.

31. (canceled)

32. The chimeric binding domain according to claim 1, wherein:
(i) the leader sequence is selected from the group consisting of Mouse Ig Kappa (METDTLLLWVLLLWVPGSTGD; SEQ ID NO:16), Human OSM (MGVLLTQRTLLSLVLALLFPSMASM; SEQ ID NO:17), VSV-G (MKCLLYLAFLFIGVNC; SEQ ID NO: 18), Human lgG2 H (MGWSCIILFLVATATGVHS; SEQ ID NO:19), BM40 (MRAWIFFLLCLAGRALA; SEQ ID NO:20), Secrecon (MWWRLWWLLLLLLLLWPMVWA; SEQ ID NO:21), Human IgKVIII (MDMRVPAQLLGLLLLWLRGARC; SEQ ID NO:22), CD33 (MPLLLLLPLLWAGALA; SEQ ID NO:23), tPA (MDAMKRGLCCVLLLCGAVFVSPS; SEQ ID NO:24), Human Chymotrypsinogen (MAFLWLLSCWALLGTTFG; SEQ ID NO:25), Human trypsinogen-2 (MNLLLILTFVAAAVA; SEQ ID NO:26), Human IL-2 (MYRMQLLSCIALSLALVTNS; SEQ ID NO:27), Gaussia luc (MGVKVLFALICIAVAEA; SEQ ID NO:28), Albumin (HSA) (MKWVTFISLLFSSAYS; SEQ ID NO:29), Influenza Haemagglutinin (MKTIIALSYIFCLVLG; SEQ ID NQ: 30), Human insulin (MALWMRLLPLLALLALWGPDPAAA; SEQ ID NO:31), Silkworm Fibroin LC and (MKPIFLVLLVVTSAYA; SEQ ID NO:32); and/or
(ii) the transmembrane domain is selected from the group consisting of the transmembrane domain of human platelet-derived growth factor receptor (PDGFR), human asialoglycoprotein receptor, human and murine B7-1, human ICAM-1, human erbbI, human erbb2, human erbb3, human erbb4, human fibroblast growth factor receptors such as FGFR 1, FGFR2, FGFR3, FGFR4, human VEGFR-1, human VEGFR-2, human erythropoietin receptor, human PRL-R, prolactin receptor, human EphA1, Ephrin type-A receptor 1, human insulin, IGF-1 receptors, human receptor-like protein tyrosine phosphatases, human neuropilin, human major histocompatibility complex class II (alpha and beta chains), human integrins (alpha and beta families), human Syndecans, human Myelin protein, human cadherins, human synaptobrevin-2, human glycophorin-A, human Bnip3, human APP, amyloid precursor protein, human T-cell receptor alpha and beta, CD3 gamma, CD3 delta, CD3 zeta, and CD3 epsilon.

33-34. (canceled)

35. A nucleic acid molecule encoding the chimeric binding domain of claim 29.

36. A pharmaceutical composition comprising the mammalian cell according to claim 1, together with a pharmaceutically acceptable carrier and/or excipient.

37. (canceled)


SEQUENCE LISTING (33 sequences)

SEQ ID NO: 1 - protein, 126 aa, synthetic construct
  VARIANT 31: note = A or Y
  VARIANT 56: note = T or M
  VARIANT 106: note = L or E
KAMHVAQPAVVLASSRGIASFVCEYASPGKATEVRVTVLRQADSQVTEVCAATYMTGNELTFLDDSICTGTSSGNQVNLTIQGLRAMDTGLYICKVELMYPPPYYLGIGNGTQIYVIDPEPSPDSN

SEQ ID NO: 2 - protein, 107 aa, synthetic construct
  VAR_SEQ 26: note = replace X with binding loop (BL) 1 = any amino acid or any number of amino acids between 5 and 15 residues inclusive
  VAR_SEQ 48: note = replace X with binding loop (BL) 2 = any amino acid and any number of amino acids between 5 and 15 residues inclusive
  VAR_SEQ 86: note = replace X with binding loop (BL) 3 = any amino acid or any number of amino acids between 5 and 15 residues inclusive
KAMHVAQPAVVLASSRGIASFVCEYXVRVTVLRQADSQVTEVCAATYXLTFLDDSICTGTSSGNQVNLTIQGLRAMDTGLYICKVXLGIGNGTQIYVIDPEPSPDSN

SEQ ID NO: 3 - protein, 8 aa, Homo sapiens
ASPGKATE

SEQ ID NO: 4 - protein, 8 aa, Homo sapiens
ASPGKYTE

SEQ ID NO: 5 - protein, 5 aa, Homo sapiens
MTGNE

SEQ ID NO: 6 - protein, 9 aa, Homo sapiens
ELMYPPPYY

SEQ ID NO: 7 - protein, 107 aa, synthetic construct
  VAR_SEQ 26: note = replace X with binding loop 1 (BL-1 )= any amino acid or any number of amino acids between 5 and 15 residues inclusive
  VAR_SEQ 48: note = replace X with binding loop 2 (BL-2) = any amino acid or any number of amino acids between 5 and 15 residues inclusive
  VAR_SEQ 86: note = replace X with binding loop 3 (BL-3) = any amino acid or any number of amino acids between 5 and 15 residues inclusive
KAMHVAQPAVVLASSRGIASFVCEYXVRVTVLRQADSQVTEVCAATYXLTFLDDSICTGTSSGNQVNLTIQGLRAMDTGLYICKVXEGIGNGTQIYVIDPEPSPDSN

SEQ ID NO: 8 - protein, 8 aa, Homo sapiens
TVSWVDME

SEQ ID NO: 9 - protein, 5 aa, Homo sapiens
WNGRW

SEQ ID NO: 10 - protein, 13 aa, Homo sapiens
QLDPSWGYYWQGY

SEQ ID NO: 11 - protein, 126 aa, synthetic construct
KAMHVAQPAVVLASSRGIASFVCEYASPGKYTEVRVTVLRQADSQVTEVCAATYMTGNELTFLDDSICTGTSSGNQVNLTIQGLRAMDTGLYICKVELMYPPPYYLGIGNGTQIYVIDPEPSPDSN

SEQ ID NO: 12 - protein, 130 aa, synthetic construct
KAMHVAQPAVVLASSRGIASFVCEYTVSWVDMEVRVTVLRQADSQVTEVCAATYWNGRWLTFLDDSICTGTSSGNQVNLTIQGLRAMDTGLYICKVQLDPSWGYYWQGYEGIGNGTQIYVIDPEPSPDSN

SEQ ID NO: 13 - protein, 5 aa, synthetic construct
SGGGG

SEQ ID NO: 14 - protein, 16 aa, synthetic construct
SGGGGSGGGGSGGGGS

SEQ ID NO: 15 - protein, 21 aa, synthetic construct
SGGGGSGGGGSGGGGSGGGGS

SEQ ID NO: 16 - protein, 21 aa, Mus musculus
METDTLLLWVLLLWVPGSTGD

SEQ ID NO: 17 - protein, 25 aa, Homo sapiens
MGVLLTQRTLLSLVLALLFPSMASM

SEQ ID NO: 18 - protein, 16 aa, Vesicularia sp.
MKCLLYLAFLFIGVNC

SEQ ID NO: 19 - protein, 19 aa, Homo sapiens
MGWSCIILFLVATATGVHS

SEQ ID NO: 20 - protein, 17 aa, Homo sapiens
MRAWIFFLLCLAGRALA

SEQ ID NO: 21 - protein, 21 aa, synthetic construct
MWWRLWWLLLLLLLLWPMVWA

SEQ ID NO: 22 - protein, 22 aa, Homo sapiens
MDMRVPAQLLGLLLLWLRGARC

SEQ ID NO: 23 - protein, 16 aa, Homo sapiens
MPLLLLLPLLWAGALA

SEQ ID NO: 24 - protein, 23 aa, Homo sapiens
MDAMKRGLCCVLLLCGAVFVSPS

SEQ ID NO: 25 - protein, 18 aa, Homo sapiens
MAFLWLLSCWALLGTTFG

SEQ ID NO: 26 - protein, 15 aa, Homo sapiens
MNLLLILTFVAAAVA

SEQ ID NO: 27 - protein, 20 aa, Homo sapiens
MYRMQLLSCIALSLALVTNS

SEQ ID NO: 28 - protein, 17 aa, Gaussia princeps
MGVKVLFALICIAVAEA

SEQ ID NO: 29 - protein, 16 aa, Homo sapiens
MKWVTFISLLFSSAYS

SEQ ID NO: 30 - protein, 16 aa, Alphainfluenzavirus influenzae
MKTIIALSYIFCLVLG

SEQ ID NO: 31 - protein, 24 aa, Homo sapiens
MALWMRLLPLLALLALWGPDPAAA

SEQ ID NO: 32 - protein, 16 aa, Bombyx sp.
MKPIFLVLLVVTSAYA

SEQ ID NO: 33 - DNA, 317 bp, synthetic construct
  source 1..317: mol_type = other DNA
  misc_difference 76: note = replace n with binding loop 1 (BL-1)
  misc_difference 140: note = replace n with binding loop 2 (BL-2)
  misc_difference 252: note = replace n with binding loop 3 (BL-3)
aaagcaatgcatgttgcacagcctgcagttgttctggcaagcagccgtggtattgccagctttgtttgtgagtatngtgcgcgtgaccgttctgcgtcaggcagatagccaggttaccgaagtttgtgcagcaacctatnctgacctttctggatgatagcatttgtaccggcaccagcagcggtaatcaggttaatctgaccattcagggtctgcgtgcaatggataccggtctgtatatttgcaaagttnctgggcattggcaatggcacccagatttatgttattgatcctgaaccgtcaccggatagcaatgc